(19) United States
(12) Patent Application Publication
(10) Pub. No.: US 2008/0194841 A1
(43) Pub. Date: Aug. 14, 2008

(54) Preparation of Optically Pure Beta-Amino Acids Having Affinity for the Alpha-Delta Protein

(75) Inventors: Paul Matthew Herrinton, South Lyon, MI (US); William Roland Perrault, Kalamazoo, MI (US); Thaddeus S. Franczyk, Lake Villa, IL (US)

Correspondence Address: PFIZER INC;Steve T. Zelson, 150 EAST 42ND STREET, 5TH FLOOR - STOP 49, NEW YORK, NY, 10017-5612, US

(73) Assignee: PFIZER INC.

(21) Appl. No.: 11/909,302

(22) PCT Filed: Mar. 13, 2006
(86) PCT No.: PCT/IB2006/000637

§ 371 Date: Sep. 21, 2007

Related U.S. Application Data
(60) Provisional application No. 60/665,502, filed on Mar. 24, 2005.

(51) Int. Cl. C07D 207/00; C07C 229/02

(52) U.S. Cl. 548/572; 562/553

(57) ABSTRACT

Disclosed are materials and methods for preparing optically active β-amino acids, which bind to the alpha-2-delta subunit of a calcium channel and are useful for treating pain, fibromyalgia, and a variety of psychiatric and sleep disorders. The method includes reacting a chiral allyl amine with a 2-alkynoate in the presence of a Lewis acid and a base to give a chiral tertiary enamine, which after reaction with ammonia, is hydrogenated to give optically active β-amino acids.

### CROSS-REFERENCE TO RELATED APPLICATION

This application claims the benefit of U.S. Provisional Patent Application No. 60/665,502, filed Mar. 24, 2005.

### BACKGROUND OF THE INVENTION

1. Field of the Invention

This invention relates to materials and methods for preparing optically-active β-amino acids that bind to the alpha-2-delta subunit of a calcium channel. The β-amino acids are useful for treating pain, fibromyalgia, and a variety of psychiatric and sleep disorders.

2. Discussion

Published U.S. Patent Application No. 2003/0195251 A1 to Barta et al. (the '251 application) and published U.S. Patent Application No. 2005/0124668 to Deur et al. (the '668 application) describe β-amino acids that bind to the α-2δ subunit of a calcium channel. These compounds, including their pharmaceutically acceptable complexes, salts, solvates, and hydrates, may be used to treat a number of disorders, conditions, and diseases, including sleep disorders, such as insomnia; fibromyalgia; epilepsy; neuropathic pain, including acute and chronic pain; migraine; hot flashes; pain associated with irritable bowel syndrome; restless leg syndrome; anorexia; panic disorder; depression; seasonal affective disorders; and anxiety, including general anxiety disorder, obsessive compulsive behavior, and attention deficit hyperactivity disorder, among others.

Many of the β-amino acids described in the '251 and '668 applications are optically active. Some of the compounds, like those represented by Formula 1 below, possess two or more stereogenic (chiral) centers, which make their preparation challenging. Although the '251 and '668 applications describe useful methods for preparing optically-active β-amino acids at laboratory bench scale, many of the methods are problematic for pilot- or full-scale production. Thus, improved methods for preparing precursors of the optically-active β-amino acids would be desirable.

### SUMMARY OF THE INVENTION

The present invention provides comparatively efficient and cost-effective methods for preparing compounds of Formula 1,

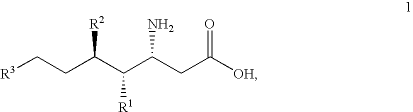

stereoisomers thereof, or pharmaceutically acceptable complexes, salts, solvates or hydrates of the compounds of Formula 1 or their stereoisomers. In Formula 1, substituents R¹, R² and R³ are each independently selected from hydrogen atom, C₁₋₆ alkyl, C₃₋₆ cycloalkyl, C₃₋₆ cycloalkyl-C₁₋₆ alkyl, aryl, aryl-C₁₋₃ alkyl, and arylamino, wherein each alkyl moiety is optionally substituted with from one to five fluorine atoms, and each aryl moiety is optionally substituted with from one to three substituents independently selected from chloro, fluoro, amino, nitro, cyano, C₁₋₃ alkylamino, C₁₋₃ alkyl optionally substituted with from one to three fluorine atoms, and C₁₋₃ alkoxy optionally substituted with from one to three fluorine atoms, provided that R¹ and R² are not both hydrogen atoms. The method comprises:

(a) reacting a compound of Formula 6,

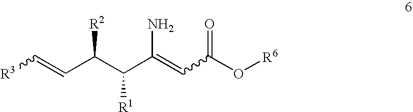

or a compound of Formula 8,

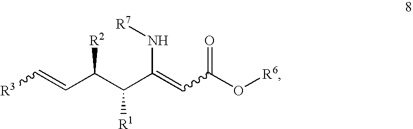

a stereoisomer of the compounds of Formula 6 or Formula 8, or a complex, salt, solvate, or hydrate of the compounds of Formula 6, Formula 8, or their stereoisomers, with H₂ in the presence of a catalyst to give a compound of Formula 9,

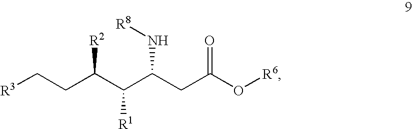

a stereoisomer thereof, or a complex, salt, solvate, or hydrate of the compound of Formula 9 or the stereoisomer thereof, wherein

R¹, R², and R³ in Formula 6, Formula 8, and Formula 9 are as defined for Formula 1;

R⁶ in Formula 6, Formula 8, and Formula 9 is a hydrogen atom, C₁₋₆ alkyl, C₂₋₆ alkenyl, C₂₋₆ alkynyl, C₃₋₇ cycloalkyl, C₃₋₇ cycloalkenyl, halo-C₁₋₇ alkyl, halo-C₂₋₇ alkenyl, halo-C₂₋₇ alkynyl, aryl-C₁₋₆ alkyl, aryl-C₂₋₆ alkenyl, or aryl-C₂₋₆ alkynyl; and

R⁷ in Formula 8 and R⁸ in Formula 9 are each independently selected from hydrogen atom, carboxy, C₁₋₇ alkanoyl, C₂₋₇ alkenoyl, C₂₋₇ alkynoyl, C₃₋₇ cycloalkanoyl, C₃₋₇ cycloalkenoyl, halo-C₁₋₇ alkanoyl, halo-C₂₋₇ alkenoyl, halo-C₂₋₇ alkynoyl, C₁₋₆ alkoxycarbonyl, halo-C₁₋₆ alkoxycarbonyl, C₃₋₇ cycloalkoxycarbonyl, aryl-C₁₋₇ alkanoyl, aryl-C₂₋₇ alkenoyl, aryl-C₂₋₇ alkynoyl, aryloxycarbonyl, and aryl-C₁₋₆ alkoxycarbonyl, provided that R⁷ is not a hydrogen atom; and

(b) optionally converting the compound of Formula 9, the stereoisomer thereof, or the complex, salt, solvate or hydrate of the compound of Formula 9 or the stereoisomer, to the compound of Formula 1, the stereoisomer thereof, or the pharmaceutically acceptable complex, salt, solvate or hydrate of the compound of Formula 1 or the stereoisomer thereof.

Another aspect of the present invention provides a method of making a compound of Formula 5,

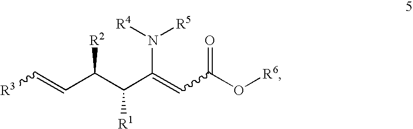

a stereoisomer thereof, or a complex, salt, solvate, or hydrate of the compound of Formula 5 or the stereoisomer thereof. The method comprises reacting a compound of Formula 2,

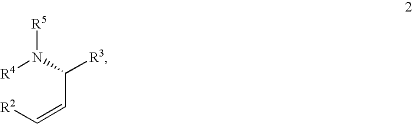

a stereoisomer thereof, or a complex, salt, solvate, or hydrate of the compound of Formula 2 or the stereoisomer thereof, with a compound of Formula 3,

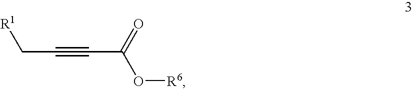

or a complex, salt, solvate, or hydrate thereof, in the presence of a Lewis acid and a base, wherein R¹, R², and R³ in Formula 2, 3, and 5 are as defined for Formula 1, above, R⁶ is as defined for Formula 6, above, and R⁴ and R⁵ are each independently selected from C₁₋₆ alkyl, or together with a nitrogen atom to which R⁴ and R⁵ are attached, form a 5- or 6-member heterocycle that may be further substituted with none, one, or two substituents selected from C₁₋₆ alkyl.

Particularly useful methods include those in which R³ is not H and the compound of Formula 2 has (R,Z)-stereochemical configuration; those in which R³ is not H, R¹ is H, and the compound of Formula 2 has (E,S)-stereochemical configuration; and those in which R³ is H, the compound of Formula 2 has (Z)-stereochemical configuration, and R⁴ and R⁵ together are (S)-2-methylpyrrolidinyl.

A further aspect of the present invention provides compounds of Formula 10,

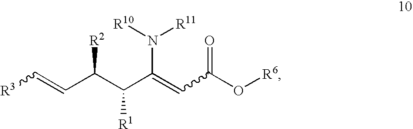

stereoisomers thereof, or complexes, salts, solvates or hydrates of the compounds of Formula 10 or stereoisomers thereof, wherein

R¹, R² and R³ are as defined above for Formula 1;

R¹⁰ and R¹¹ are each independently selected from hydrogen atom, C₁₋₆ alkyl, carboxy, C₁₋₇ alkanoyl, C₂₋₇ alkenoyl, C₂₋₇ alkynoyl, C₃₋₇ cycloalkanoyl, C₃₋₇ cycloalkenoyl, halo-C₁₋₇ alkanoyl, halo-C₂₋₇ alkenoyl, halo-C₂₋₇ alkynoyl, C₁₋₆ alkoxycarbonyl, halo-C₁₋₆ alkoxycarbonyl, C₃₋₇ cycloalkoxycarbonyl, aryl-C₁₋₇ alkanoyl, aryl-C₂₋₇ alkenoyl, aryl-C₂₋₇ alkynoyl, aryloxycarbonyl, and aryl-C₁₋₆ alkoxycarbonyl, or together with a nitrogen atom to which R¹⁰ and R¹¹ are attached, form a 5- or 6-member heterocycle that may be further substituted with none, one, or two substituents selected from C₁₋₆ alkyl; and

R⁶ is as defined above for Formula 6.

The compounds of Formula 10 include those given by Formula 5, Formula 6, and Formula 8, above, as well as those given by the following compounds and their complexes, salts, solvates, hydrates, and C₁₋₄ alkyl esters (e.g., Me, Et, i-Pr, n-Pr, n-Bu, i-Bu, s-Bu, and t-Bu):

(2S,5S)-5-methyl-3-(2-methyl-pyrrolidin-1-yl)-hepta-2,6-dienoic acid;

(S)-5-methyl-3-pyrrolidin-1-yl-octa-2,6-dienoic acid;

(S)-5-methyl-3-pyrrolidin-1-yl-nona-2,6-dienoic acid;

(S)-3-amino-5-methyl-hepta-2,6-dienoic acid;

(S)-3-amino-5-methyl-octa-2,6-dienoic acid;

(S)-3-amino-5-methyl-nona-2,6-dienoic acid;

(S)-3-acetylamino-5-methyl-hepta-2,6-dienoic acid;

(S)-3-acetylamino-5-methyl-octa-2,6-dienoic acid;

(S)-3-acetylamino-5-methyl-nona-2,6-dienoic acid;

(2S,4R,5R)-4,5-dimethyl-3-(2-methyl-pyrrolidin-1-yl)-hepta-2,6-dienoic acid;

(R,R)-4,5-dimethyl-3-pyrrolidin-1-yl-octa-2,6-dienoic acid;

(R,R)-4,5-dimethyl-3-pyrrolidin-1-yl-nona-2,6-dienoic acid;

(R,R)-3-amino-4,5-dimethyl-hepta-2,6-dienoic acid;

(R,R)-3-amino-4,5-dimethyl-octa-2,6-dienoic acid;

(R,R)-3-amino-4,5-dimethyl-nona-2,6-dienoic acid;

(R,R)-3-acetylamino-4,5-dimethyl-hepta-2,6-dienoic acid;

(R,R)-3-acetylamino-4,5-dimethyl-octa-2,6-dienoic acid;

(R,R)-3-acetylamino-4,5-dimethyl-nona-2,6-dienoic acid; and opposite enantiomers and diastereomers of the aforementioned compounds.

Certain compounds may contain an alkenyl or cyclic group, so that cis/trans (or Z/E) stereoisomers are possible, or may contain a keto or oxime group, so that tautomerism may occur. In such cases, the present invention generally includes all Z/E isomers and tautomeric forms, whether they are pure, substantially pure, or mixtures. The present invention includes all complexes, salts, solvates, and hydrates, whether pharmaceutically acceptable or not, and all polymorphic (crystalline and amorphous) forms of the disclosed and recited compounds and their stereoisomers, including opposite enantiomers, diastereomers, and geometrical isomers. The phrase “complexes, salts, solvates, and hydrates thereof” refers to the recited compounds and to their stereoisomers.

### DETAILED DESCRIPTION

### Definitions and Abbreviations

Unless otherwise indicated, this disclosure uses definitions provided below. Some of the definitions and formulae may include a dash (“-”) to indicate a bond between atoms or a point of attachment to a named or unnamed atom or group of atoms. Other definitions and formulae may include an equal sign (“=”) or an identity symbol (“≡”) to indicate a double bond or a triple bond, respectively. Certain formulae may also include one or more asterisks (“*”) to indicate stereogenic (asymmetric or chiral) centers, although the absence of an asterisk does not indicate that the compound lacks a stereocenter. Such formulae may refer to the racemate or to individual enantiomers or to individual diastereomers, which may or may not be pure or substantially pure. Other formulae may include one or more wavy bonds ( [symbol] ). When attached to a stereogenic center, the wavy bonds refer to both stereoisomers, either individually or as mixtures. Likewise, when attached to a double bond, the wavy bonds indicate a Z-isomer, an E-isomer, or a mixture of Z and E isomers.

“Substituted” groups are those in which one or more hydrogen atoms have been replaced with one or more non-hydrogen atoms or groups, provided that valence requirements are met and that a chemically stable compound results from the substitution.

“About” or “approximately,” when used in connection with a measurable numerical variable, refers to the indicated value of the variable and to all values of the variable that are within the experimental error of the indicated value (e.g., within the 95% confidence interval for the mean) or within ±10 percent of the indicated value, whichever is greater.

“Alkyl” refers to straight chain and branched saturated hydrocarbon groups, generally having a specified number of carbon atoms (i.e., C₁₋₆ alkyl refers to an alkyl group having 1, 2, 3, 4, 5, or 6 carbon atoms). Examples of alkyl groups include methyl, ethyl, n-propyl, i-propyl, n-butyl, s-butyl, i-butyl, t-butyl, pent-1-yl, pent-2-yl, pent-3-yl, 3-methylbut-1-yl, 3-methylbut-2-yl, 2-methylbut-2-yl, 2,2,2-trimethyleth-1-yl, n-hexyl, and the like.

“Alkenyl” refers to straight chain and branched hydrocarbon groups having one or more unsaturated carbon-carbon bonds, and generally having a specified number of carbon atoms. Examples of alkenyl groups include ethenyl, 1-propen-1-yl, 1-propen-2-yl, 2-propen-1-yl, 1-buten-1-yl, 1-buten-2-yl, 3-buten-1-yl, 3-buten-2-yl, 2-buten-1-yl, 2-buten-2-yl, 2-methyl-1-propen-1-yl, 2-methyl-2-propen-1-yl, 1,3-butadien-1-yl, 1,3-butadien-2-yl, and the like.

“Alkynyl” refers to straight chain or branched hydrocarbon groups having one or more triple carbon-carbon bonds, and generally having a specified number of carbon atoms. Examples of alkynyl groups include ethynyl, 1-propyn-1-yl, 2-propyn-1-yl, 1-butyn-1-yl, 3-butyn-1-yl, 3-butyn-2-yl, 2-butyn-1-yl, and the like.

“Alkanoyl” refers to alkyl-C(O)—, where alkyl is defined above, and generally includes a specified number of carbon atoms, including the carbonyl carbon. Examples of alkanoyl groups include formyl, acetyl, propionyl, butyryl, pentanoyl, hexanoyl, and the like.

“Alkenoyl” and “alkynoyl” refer, respectively, to alkenyl-C(O)— and alkynyl-C(O)—, where alkenyl and alkynyl are defined above. References to alkenoyl and alkynoyl generally include a specified number of carbon atoms, excluding the carbonyl carbon. Examples of alkenoyl groups include propenoyl, 2-methylpropenoyl, 2-butenoyl, 3-butenoyl, 2-methyl-2-butenoyl, 2-methyl-3-butenoyl, 3-methyl-3-butenoyl, 2-pentenoyl, 3-pentenoyl, 4-pentenoyl, and the like. Examples of alkynoyl groups include propynoyl, 2-butynoyl, 3-butynoyl, 2-pentynoyl, 3-pentynoyl, 4-pentynoyl, and the like.

“Alkoxy” and “alkoxycarbonyl” refer, respectively, to alkyl-O—, alkenyl-O, and alkynyl-O, and to alkyl-O—C(O)—, alkenyl-O—C(O)—, alkynyl-O—C(O)—, where alkyl, alkenyl, and alkynyl are defined above. Examples of alkoxy groups include methoxy, ethoxy, n-propoxy, i-propoxy, n-butoxy, s-butoxy, t-butoxy, n-pentoxy, s-pentoxy, and the like. Examples of alkoxycarbonyl groups include methoxycarbonyl, ethoxycarbonyl, n-propoxycarbonyl, i-propoxycarbonyl, n-butoxycarbonyl, s-butoxycarbonyl, t-butoxycarbonyl, n-pentoxycarbonyl, s-pentoxycarbonyl, and the like.

“Halo,” “halogen” and “halogeno” may be used interchangeably, and refer to fluoro, chloro, bromo, and iodo.

“Haloalkyl,” “haloalkenyl,” “haloalkynyl,” “haloalkanoyl,” “haloalkenoyl,” “haloalkynoyl,” “haloalkoxy,” and “haloalkoxycarbonyl” refer, respectively, to alkyl, alkenyl, alkynyl, alkanoyl, alkenoyl, alkynoyl, alkoxy, and alkoxycarbonyl groups substituted with one or more halogen atoms, where alkyl, alkenyl, alkynyl, alkanoyl, alkenoyl, alkynoyl, alkoxy, and alkoxycarbonyl are defined above. Examples of haloalkyl groups include trifluoromethyl, trichloromethyl, pentafluoroethyl, pentachloroethyl, and the like.

“Cycloalkyl” refers to saturated monocyclic and bicyclic hydrocarbon rings, generally having a specified number of carbon atoms that comprise the ring (i.e., C₃₋₇ cycloalkyl refers to a cycloalkyl group having 3, 4, 5, 6 or 7 carbon atoms as ring members). The cycloalkyl may be attached to a parent group or to a substrate at any ring atom, unless such attachment would violate valence requirements. Likewise, any of the ring members may include one or more non-hydrogen substituents unless such substitution would violate valence requirements. Useful substituents include alkyl, alkenyl, alkynyl, haloalkyl, haloalkenyl, haloalkynyl, alkoxy, alkoxycarbonyl, alkanoyl, and halo, as defined above, and hydroxy, mercapto, nitro, and amino.

Examples of monocyclic cycloalkyl groups include cyclopropyl, cyclobutyl, cyclopentyl, cyclohexyl, and the like. Examples of bicyclic cycloalkyl groups include bicyclo[1.1.0]butyl, bicyclo[1.1.1]pentyl, bicyclo[2.1.0]pentyl, bicyclo[2.1.1]hexyl, bicyclo[3.1.0]hexyl, bicyclo[2.2.1]heptyl, bicyclo[3.2.0]heptyl, bicyclo[3.1.1]heptyl, bicyclo[4.1.0]heptyl, bicyclo[2.2.2joctyl, bicyclo[3.2.1]octyl, bicyclo[4.1.1]octyl, bicyclo[3.3.0]octyl, bicyclo[4.2.0]octyl, bicyclo[3.3.1]nonyl, bicyclo[4.2.1]nonyl, bicyclo[4.3.0]nonyl, bicyclo[3.3.2]decyl, bicyclo[4.2.2]decyl, bicyclo[4.3.1]decyl, bicyclo[4.4.0]decyl, bicyclo[3.3.3]undecyl, bicyclo[4.3.2]undecyl, bicyclo[4.3.3]dodecyl, and the like.

“Cycloalkenyl” refers monocyclic and bicyclic hydrocarbon rings having one or more unsaturated carbon-carbon bonds and generally having a specified number of carbon atoms that comprise the ring (i.e., C₃₋₇ cycloalkenyl refers to a cycloalkenyl group having 3, 4, 5, 6 or 7 carbon atoms as ring members). The cycloalkenyl may be attached to a parent group or to a substrate at any ring atom, unless such attachment would violate valence requirements. Likewise, any of the ring members may include one or more non-hydrogen substituents unless such substitution would violate valence requirements. Useful substituents include alkyl, alkenyl, alkynyl, haloalkyl, haloalkenyl, haloalkynyl, alkoxy, alkoxycarbonyl, alkanoyl, and halo, as defined above, and hydroxy, mercapto, nitro, and amino.

“Cycloalkanoyl” and “cycloalkenoyl” refer to cycloalkyl-C(O)— and cycloalkenyl-C(O)—, respectively, where cycloalkyl and cycloalkenyl are defined above. References to cycloalkanoyl and cycloalkenoyl generally include a specified number of carbon atoms, excluding the carbonyl carbon. Examples of cycloalkanoyl groups include cyclopropanoyl, cyclobutanoyl, cyclopentanoyl, cyclohexanoyl, cycloheptanoyl, 1-cyclobutenoyl, 2-cyclobutenoyl, 1-cyclopentenoyl, 2-cyclopentenoyl, 3-cyclopentenoyl, 1-cyclohexenoyl, 2-cyclohexenoyl, 3-cyclohexenoyl, and the like.

“Cycloalkoxy” and “cycloalkoxycarbonyl” refer, respectively, to cycloalkyl-O— and cycloalkenyl-O and to cycloalkyl-O—C(O)— and cycloalkenyl-O—C(O)—, where cycloalkyl and cycloalkenyl are defined above. References to cycloalkoxy and cycloalkoxycarbonyl generally include a specified number of carbon atoms, excluding the carbonyl carbon. Examples of cycloalkoxy groups include cyclopropoxy, cyclobutoxy, cyclopentoxy, cyclohexoxy, 1-cyclobutenoxy, 2-cyclobutenoxy, 1-cyclopentenoxy, 2-cyclopentenoxy, 3-cyclopentenoxy, 1-cyclohexenoxy, 2-cyclohexenoxy, 3-cyclohexenoxy, and the like. Examples of cycloalkoxycarbonyl groups include cyclopropuxycarbonyl, cyclobutoxycarbonyl, cyclopentoxycarbonyl, cyclohexoxycarbonyl, 1-cyclobutenoxycarbonyl, 2-cyclobutenoxycarbonyl, 1-cyclopentenoxycarbonyl, 2-cyclopentenoxycarbonyl, 3-cyclopentenoxycarbonyl, 1-cyclohexenoxycarbonyl, 2-cyclohexenoxycarbonyl, 3-cyclohexenoxycarbonyl, and the like.

“Aryl” and “arylene” refer to monovalent and divalent aromatic groups, respectively, including 5- and 6-membered monocyclic aromatic groups that contain 0 to 4 heteroatoms independently selected from nitrogen, oxygen, and sulfur. Examples of monocyclic aryl groups include phenyl, pyrrolyl, furanyl, thiopheneyl, thiazolyl, isothiazolyl, imidazolyl, triazolyl, tetrazolyl, pyrazolyl, oxazolyl, isooxazolyl, pyridinyl, pyrazinyl, pyridazinyl, pyrimidinyl, and the like. Aryl and arylene groups also include bicyclic groups, tricyclic groups, etc., including fused 5- and 6-membered rings described above. Examples of multicyclic aryl groups include naphthyl, biphenyl, anthracenyl, pyrenyl, carbazolyl, benzoxazolyl, benzodioxazolyl, benzothiazolyl, benzoimidazolyl, benzothiopheneyl, quinolinyl, isoquinolinyl, indolyl, benzofuranyl, purinyl, indolizinyl, and the like. The aryl and arylene groups may be attached to a parent group or to a substrate at any ring atom, unless such attachment would violate valence requirements. Likewise, any of the carbon or nitrogen ring members may include a non-hydrogen substituent unless such substitution would violate valence requirements. Useful substituents include alkyl, alkenyl, alkynyl, haloalkyl, haloalkenyl, haloalkynyl, cycloalkyl, cycloalkenyl, alkoxy, cycloalkoxy, alkanoyl, cycloalkanoyl, cycloalkenoyl, alkoxycarbonyl, cycloalkoxycarbonyl, and halo, as defined above, and hydroxy, mercapto, nitro, amino, and alkylamino.

“Heterocycle” and “heterocyclyl” refer to saturated, partially unsaturated, or unsaturated monocyclic or bicyclic rings having from 5 to 7 or from 7 to 11 ring members, respectively. These groups have ring members made up of carbon atoms and from 1 to 4 heteroatoms that are independently nitrogen, oxygen or sulfur, and may include any bicyclic group in which any of the above-defined monocyclic heterocycles are fused to a benzene ring. The nitrogen and sulfur heteroatoms may optionally be oxidized. The heterocyclic ring may be attached to a parent group or to a substrate at any heteroatom or carbon atom unless such attachment would violate valence requirements. Likewise, any of the carbon or nitrogen ring members may include a non-hydrogen substituent unless such substitution would violate valence requirements. Useful substituents include alkyl, alkenyl, alkynyl, haloalkyl, haloalkenyl, haloalkynyl, cycloalkyl, cycloalkenyl, alkoxy, cycloalkoxy, alkanoyl, cycloalkanoyl, cycloalkenoyl, alkoxycarbonyl, cycloalkoxycarbonyl, and halo, as defined above, and hydroxy, mercapto, nitro, amino, and alkylamino.

Examples of heterocycles include acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, inidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl.

“Heteroaryl” and “heteroarylene” refer, respectively, to monovalent and divalent heterocycles or heterocyclyl groups, as defined above, which are aromatic. Heteroaryl and heteroarylene groups represent a subset of aryl and arylene groups, respectively.

“Arylalkyl” and “heteroarylalkyl” refer, respectively, to aryl-alkyl and heteroaryl-alkyl, where aryl, heteroaryl, and alkyl are defined above. Examples include benzyl, fluorenylmethyl, imidazol-2-yl-methyl, and the like.

“Arylalkanoyl,” “heteroarylalkanoyl,” “arylalkenoyl,” “heteroarylalkenoyl,” “arylalkynoyl,” and “heteroarylalkynoyl” refer, respectively, to aryl-alkanoyl, heteroaryl-alkanoyl, aryl-alkenoyl, heteroaryl-alkenoyl, aryl-alkynoyl, and heteroaryl-alkynoyl, where aryl, heteroaryl, alkanoyl, alkenoyl, and alkynoyl are defined above. Examples include benzoyl, benzylcarbonyl, fluorenoyl, fluorenylmethylcarbonyl, imidazol-2-oyl, imidazol-2-yl-methylcarbonyl, phenylethenecarbonyl, 1-phenylethenecarbonyl, 1-phenyl-propenecarbonyl, 2-phenyl-propenecarbonyl, 3-phenyl-propenecarbonyl, imidazol-2-yl-ethenecarbonyl, 1-(imidazol-2-yl)-ethenecarbonyl, 1-(imidazol-2-yl)-propenecarbonyl, 2-(imidazol-2-yl)-propenecarbonyl, 3-(imidazol-2-yl)-propenecarbonyl, phenylethynecarbonyl, phenylpropynecarbonyl, (imidazol-2-yl)-ethynecarbonyl, (imidazol-2-yl)-propynecarbonyl, and the like.

“Arylalkoxy” and “heteroarylalkoxy” refer, respectively, to aryl-alkoxy and heteroaryl-alkoxy, where aryl, heteroaryl, and alkoxy are defined above. Examples include benzyloxy, fluorenylmethyloxy, imidazol-2-yl-methyloxy, and the like.

“Aryloxy” and “heteroaryloxy” refer, respectively, to aryl-O— and heteroaryl-O—, where aryl and heteroaryl are defined above. Examples include phenoxy, imidazol-2-yloxy, and the like.

“Aryloxycarbonyl,” “heteroaryloxycarbonyl,” “arylalkoxycarbonyl,” and “heteroarylalkoxycarbonyl” refer, respectively, to aryloxy-C(O)—, heteroaryloxy-C(O)—, arylalkoxy-C(O)—, and heteroarylalkoxy-C(O)—, where aryloxy, heteroaryloxy, arylalkoxy, and heteroarylalkoxy are defined above. Examples include phenoxycarbonyl, imidazol-2-yloxycarbonyl, benzyloxycarbonyl, fluorenylmethyloxycarbonyl, imidazol-2-yl-methyloxycarbonyl, and the like.

“Leaving group” refers to any group that leaves a molecule during a fragmentation process, including substitution reactions, elimination reactions, and addition-elimination reactions. Leaving groups may be nucleofugal, in which the group leaves with a pair of electrons that formerly served as the bond between the leaving group and the molecule, or may be electrofugal, in which the group leaves without the pair of electrons. The ability of a nucleofugal leaving group to leave depends on its base strength, with the strongest bases being the poorest leaving groups. Common nucleofugal leaving groups include nitrogen (e.g., from diazonium salts); sulfonates, including alkylsulfonates (e.g., mesylate), fluoroalkylsulfonates (e.g., triflate, hexaflate, nonaflate, and tresylate), and arylsulfonates (e.g., tosylate, brosylate, closylate, and nosylate). Others include carbonates, halide ions, carboxylate anions, phenolate ions, and alioxides. Some stronger bases, such as NH₂⁻ and OH⁻ can be made better leaving groups by treatment with an acid. Common electrofugal leaving groups include the proton, CO₂, and metals.

“Enantiomeric excess” or “ee” is a measure, for a given sample, of the excess of one enantiomer over a racemic sample of a chiral compound and is expressed as a percentage. Enantiomeric excess is defined as 100×(er−1)/(er+1), where “er” is the ratio of the more abundant enantiomer to the less abundant enantiomer.

“Diastereomeric excess” or “de” is a measure, for a given sample, of the excess of one diastereomer over a sample having equal amounts of diastereomers and is expressed as a percentage. Diastereomeric excess is defined as 100×(dr−1)/(dr+1), where “dr” is the ratio of a more abundant diastereomer to a less abundant diastereomer.

“Stereoselective,” “enantioselective,” “diastereoselective,” and variants thereof, refer to a given process (e.g., hydrogenation) that yields more of one stereoisomer, enantiomer, or diastereoisomer than of another, respectively.

“High level of stereoselectivity,” “high level of enantioselectivity,” “high level of diastereoselectivity,” and variants thereof, refer to a given process that yields products having an excess of one stereoisomer, enantiomer, or diastereoisomer, which comprises at least about 90% of the products. For a pair of enantiomers or diastereomers, a high level of enantioselectivity or diastereoselectivity would correspond to an cc or de of at least about 80%.

“Stereoisomerically enriched,” “enantiomerically enriched,” “diastereomerically enriched,” and variants thereof, refer, respectively, to a sample of a compound that has more of one stereoisomer, enantiomer or diastereomer than another. The degree of enrichment may be measured by % of total product, or for a pair of enantiomers or diastereomers, by ee or de.

“Substantially pure stereoisomer,” “substantially pure enantiomer,” “substantially pure diastereomer,” and variants thereof, refer, respectively, to a sample containing a stereoisomer, enantiomer, or diastereomer, which comprises at least about 95% of the sample. For pairs of enantiomers and diastereomers, a substantially pure enantiomer or diastereomer would correspond to samples having an ee or de of about 90% or greater.

A “pure stereoisomer,” “pure enantiomer,” “pure diastereomer,” and variants thereof, refer, respectively, to a sample containing a stereoisomer, enantiomer, or diastereomer, which comprises at least about 99.5% of the sample. For pairs of enantiomers and diastereomers, a pure enantiomer or pure diastereomer” would correspond to samples having an ee or de of about 99% or greater.

“Opposite enantiomer” refers to a molecule that is a non-superimposable mirror image of a reference molecule, which may be obtained by inverting all of the stereogenic centers of the reference molecule. For example, if the reference molecule has S absolute stereochemical configuration, then the opposite enantiomer has R absolute stereochemical configuration. Likewise, if the reference molecule has S,S absolute stereochemical configuration, then the opposite enantiomer has R,R stereochemical configuration, and so on.

“Stereoisomers” of a specified compound refer to the opposite enantiomer of the compound and to any diastereoisomers, including geometrical isomers (Z/E) of the compound. For example, if the specified compound has S,R,Z stereochemical configuration, its stereoisomers would include its opposite enantiomer having R,S,Z configuration, and its diastereomers having S,S,Z configuration, R,R,Z configuration, as well as S,R,E configuration, R,S,E configuration, S,S,E configuration, and R,R,E configuration.

“Solvate” refers to a molecular complex comprising a disclosed or claimed compound and a stoichiometric or non-stoichiometric amount of one or more solvent molecules (e.g., EtOH).

“Hydrate” refers to a solvate comprising a disclosed or claimed compound and a stoichiometric or non-stoichiometric amount of water.

“Pharmaceutically acceptable complexes, salts, solvates, or hydrates” refers to complexes, acid or base addition salts, solvates or hydrates of claimed and disclosed compounds, which are within the scope of sound medical judgment, suitable for use in contact with the tissues of patients without undue toxicity, irritation, allergic response, and the like, commensurate with a reasonable benefit/risk ratio, and effective for their intended use.

“Pre-catalyst” or “catalyst precursor” refers to a compound or set of compounds that are converted into a catalyst prior to use.

“Treating” refers to reversing, alleviating, inhibiting the progress of, or preventing a disorder or condition to which such term applies, or to preventing one or more symptoms of such disorder or condition.

“Treatment” refers to the act of “treating,” as defined immediately above.

Table 1 lists abbreviations used throughout the specification.

TABLE 1
List of Abbreviations

| Abbreviation | Description |
|---|---|
| Ac | acetyl |
| ACN | acetonitrile |
| Ac₂O | acetic anhydride |
| aq | aqueous |
| (R,R)-BDPP | (2R,4R)-(+)-2,4-bis(diphenylphosphino)pentane |
| (R)-BICHEP | (R)-(−)-2,2′-bis(dicyclohexylphosphino)-6,6′-dimethyl-1,1′- |
| | biphenyl |
| (S,S)-BICP | (2S,2′S)-bis(diphenylphosphino)-(1S,1′S)-bicyclopentane |
| BIFUP | 2,2′-bis(diphenylphosphino)-4,4′,6,6′- |
| | tetrakis(trifluoromethyl)-1,1′-biphenyl |
| (R)-Tol-BINAP | (R)-(+)-2,2′-bis(di-p-tolylphosphino)-1,1′-binaphthyl |
| (S)-Tol-BINAP | (S)-(+)-2,2′-bis(di-p-tolylphosphino)-1,1′-binaphthyl |
| (R)-BINAP | (R)-2,2′-bis(diphenylphosphino)-1′1-binaphthyl |
| (S)-BINAP | (S)-2,2′-bis(diphenylphosphino)-1′1-binaphthyl |
| BIPHEP | 2,2′-bis(diphenylphosphino)-1,1′-biphenyl |
| (R)-MeO-BIPHEP | (R)-(6,6′-dimethoxybiphenyl-2,2′-diyl)- |
| | bis(diphenylphosphine) |
| (R)—Cl-MeO-BIPHEP | (R)-(+)-5,5′-dichloro-6,6′-dimethoxy-2,2′- |
| | bis(diphenylphosphino)-1,1′-biphenyl |
| (S)—Cl-MeO-BIPHEP | (S)-(+)-5,5′-dichloro-6,6′-dimethoxy-2,2′- |
| | bis(diphenylphosphino)-1,1′-biphenyl |
| BisP* | (S,S)-1,2-bis(t-butylmethylphosphino)ethane |
| (+)-tetraMeBITIANP | (S)-(+)-2,2′-bis(diphenylphosphino)-4,4′,6,6′-tetramethyl- |
| | 3,3′-bibenzo[b]thiophene |
| Bn | benzyl |
| BnBr, BnCl | benzylbromide, benzylchloride |
| Boc | t-butoxycarbonyl |
| (R)-BINAPINE | (3R,3′R,4R,4′R,11bS,11′bS)-4,4′-bis(1,1-dimethylethyl)- |
| | 4,4′,5,5′-tetrahydro-3,3′-bi-3H-dinaphtho[2,1-c:1′,2′- |
| | e]phosphepin-κP4,κP4′ |
| BOP | benzotriazol-1-yloxy-tris-(dimethylamino)-phosphonium |
| | hexafluorophosphate |
| bp | normal boiling point |
| (R)-(S)-BPPFA | (−)-(R)-N,N-dimethyl-1-((S)-1′,2- |
| | bis(diphenylphosphino)ferrocenyl)ethylamine |
| (R,R)-Et-BPE | (+)-1,2-bis((2R,5R)-2,5-diethylphospholano)ethane |
| (R,R)-Me-BPE | (+)-1,2-bis((2R,5R)-2,5-dimethylphospholano)ethane |
| (S,S)-BPPM | (−)-(2S,4S)-2-diphenylphosphinomethyl-4- |
| | diphenylphosphino-1-t-butoxycarbonylpyrrolidine |
| Bs | brosyl or p-bromo-benzenesulfonyl |
| Bu | butyl |
| i-Bu, n-Bu, s-Bu, t-Bu | iso-, normal, secondary, and tertiary butyl |
| n-BuLi | n-butyl lithium |
| t-BuOK | potassium tertiary-butoxide |
| t-BuOLi | lithium tertiary-butoxide |
| (+)-CAMP | (R)-(+)-cyclohexyl(2-anisyl)methylphosphine; a |
| | monophosphine |
| CARBOPHOS | methyl-α-D-glucopyranoside-2,6-dibenzoate-3,4-di(bis(3,5- |
| | dimethylphenyl)phosphinite) |
| Cbz | benzyloxycarbonyl |
| CDI | N,N-carbonyldiimidazole |
| (S,S)-CHIRAPHOS | (2S,3S)-(−)-bis(diphenylphosphino)butane |
| CnTunaPHOS | 2,2′-bis-diphenylphosphanyl-biphenyl having an —O—(CH₂)ₙ—O— |
| | group linking the 6,6′ carbon atoms of the |
| | biphenyl (e.g., (R)-1,14-bis-diphenylphosphanyl-6,7,8,9- |
| | tetrahydro-5,10-dioxa-dibenzo[a,c]cyclodecene for n = 4). |
| COD | 1,5-cyclooctadiene |
| (R)-CYCPHOS | (R)-1,2-bis(diphenylphosphino)-1-cyclohexylethane |
| DBAD | di-t-butyl azodicarboxylate |
| DBN | 1,5-diazabicyclo[4.3.0]nona-5-ene |
| DBU | 1,8-diazabicyclo[5.4.0]undec-7-ene |
| DCC | dicycohexylcarbodiimide |
| de | diastereomeric excess |
| DEAD | diethyl azodicarboxylate |
| (R,R)-DEGUPHOS | N-benzyl-(3R,4R)-3,4-bis(diphenylphosphino)pyrrolidine |
| DIAD | diisopropyl azodicarboxylate |
| (R,R)-DIOP | (4R,5R)-(−)-O-isopropylidene-2,3-dihydroxy-1,4- |
| | bis(diphenylphosphino)butane |
| (R,R)-DIPAMP | (R,R)-(−)-1,2-bis[(O- |
| | methoxyphenyl)(phenyl)phosphino]ethane |
| DIPEA | N,N′-diisopropylethylamine |
| DMAP | 4-(dimethylamino) pyridine |
| DMF | dimethylformamide |
| DMSO | dimethylsulfoxide |
| DMT-MM | 4-(4,6-dimethoxy-1,3,5-triazin-2-yl)-4-methylmorpholinium |
| | chloride |
| DSC | differential scanning calorimetry |
| (R,R)-Et-DUPHOS | (−)-1,2-bis((2R,5R)-2,5-diethylphospholano)benzene |
| (S,S)-Et-DUPHOS | (−)-1,2-bis((2S,5S)-2,5-diethylphospholano)benzene |
| (R,R)-i-Pr-DUPHOS | (+)-1,2-bis((2R,5R)-2,5-di-i-propylphospholano)benzene |
| (R,R)-Me-DUPHOS | (−)-1,2-bis((2R,5R)-2,5-dimethylphospholano)benzene |
| (S,S)-Me-DUPHOS | (−)-1,2-bis((2S,5S)-2,5-dimethylphospholano)benzene |
| EDCI | 1-(3-dimethylaminopropyl)-3-ethylcarbodiimide |
| ee | enantiomeric excess |
| Et | ethyl |
| Et₃N | triethyl-amine |
| EtOAc | ethyl acetate |
| Et₂O | diethyl ether |
| EtOH | ethyl alcohol |
| FDPP | pentafluorophenyl diphenylphosphinate |
| (R,R)-Et-FerroTANE | 1,1′-bis((2R,4R)-2,4-diethylphosphotano)ferrocene |
| Fmoc | 9-fluoroenylmethoxycarbonyl |
| GC | gas chromatography |
| h, min, s | hour(s), minute(s), second(s) |
| HOAc | acetic acid |
| HOAt | 1-hydroxy-7-azabenzotriazole |
| HOBt | N-hydroxybenzotriazole |
| HODhbt | 3-hydroxy-3,4-dihydro-4-oxo-1,2,3-benzotriazine |
| ID | internal diameter |
| (R)—(R)-JOSIPHOS | (R)-(−)-1-[(R)-2- |
| | (diphenylphosphino)ferrocenyl]ethyldicyclohexylphosphine |
| (S)—(S)-JOSIPHOS | (S)-(−)-1-[(S)-2- |
| | (diphenylphosphino)ferrocenyl]ethyldicyclohexylphosphine |
| (R)—(S)-JOSIPHOS | (R)-(−)-1-[(S)-2- |
| | (diphenylphosphino)ferrocenyl]ethyldicyclohexylphosphine |
| KHMDS | potassium hexamethyldisilazane |
| LAH | Lithium aluminum hydride |
| LDA | lithium diisopropylamide |
| LHMDS | lithium hexamethyldisilazane |
| LICA | lithium isopropylcyclohexylamide |
| LTMP | 2,2,6,6-tetramethylpiperidine |
| Me | methyl |
| MeCl₂ | methylene chloride |
| MEK | methylethylketone or butan-2-one |
| MeOH | methyl alcohol |
| (R,R)-t-butyl-miniPHOS | (R,R)-1,2-bis(di-t-butylmethylphosphino)methane |
| (S,S) MandyPhos | (S,S)-(−)-2,2′-bis[(R)-(N,N-dimethylamino) (phenyl)methyl]- |
| | 1,1′-bis(diphenylphosphino)ferrocene |
| (R)-MonoPhos | (R)-(−)-[4,N,N-dimethylamino]dinaphtho[2,1-d:1′,2′- |
| | f][1,3,2]dioxaphosphepin |
| (R)-MOP | (R)-(+)-2-(diphenylphosphino)-2′-methoxy-1,1′-binaphthyl |
| MPa | mega Pascals |
| mp | melting point |
| Ms | mesyl or methanesulfonyl |
| MTBE | methyl tertiary butyl ether |
| NBD | 2,5-norbornadiene |
| NMP | N-methylpyrrolidone |
| Ns | nosyl or nitrobenzene sulfonyl |
| (R,R)-NORPHOS | (2R,3R)-(−)-2,3-bis(diphenylphosphino)bicyclo[2.2.1]hepta- |
| | 5-ene |
| PdCl₂(dppf)₂ | dichloro[1,1′-bis(diphenylphosphino)ferrocene]palladium |
| | (II) dichloromethane adduct |
| (R,S,R,S)-Me- | (1R,2S,4R,5S)-2,5-dimethyl-7-phosphadicyclo[2.2.1]heptane |
| PENNPHOS | |
| Ph | phenyl |
| Ph₃P | triphenylphosphine |
| Ph₃As | triphenylarsine |
| (R)-PHANEPHOS | (R)-(−)-4,12-bis(diphenylphosphino)-[2.2]-paracyclophane |
| (S)-PHANEPHOS | (S)-(−)-4,12-bis(diphenylphosphino)-[2.2]-paracyclophane |
| (R)-PNNP | N,N′-bis[(R)-(+)-α-methylbenzyl]-N,N′- |
| | bis(diphenylphosphino)ethylene diamine |
| PPh₂-PhOx-Ph | (R)-(−)-2-[2-(diphenylphosphino)phenyl]-4-phenyl-2- |
| | oxazoline |
| Pr | propyl |
| i-Pr, n-Pr | isopropyl, normal propyl |
| (R)-PROPHOS | (R)-(+)-1,2-bis(diphenylphosphino)propane |
| PyBOP | benzotriazole-1-yl-oxy-tris-pyrrolidino-phosphonium |
| | hexafluorophosphate |
| (R)-QUINAP | (R)-(+)-1-(2-diphenylphosphino-1-naphthyl)isoquinoline |
| R_{f} | retention factor |
| RT | room temperature (approximately 20° C. to 25° C.) |
| s/c | substrate-to-catalyst molar ratio |
| (R)-SpirOP | (1R,5R,6R)-spiro[4.4]nonane-1,6-diyl-diphenylphosphinous |
| | acid ester; a spirocyclic phosphinite ligand |
| (R,R,S,S) TangPhos | (R,R,S,S) 1,1′-di-t-butyl-[2,2′]biphospholanyl |
| T_{det}, Tᵢₙᵢ, Tᵢₙⱼ | detector, initial, and injector temperatures |
| TATU | O-(7-azabenzotriazol-1-yl)-1,1,3,3-tetramethyluronium |
| | tetrafluoroborate |
| (R)-eTCFP | (R)-2-{[(di-t-butyl-phosphanyl)-ethyl]-methyl-phosphanyl}- |
| | 2-methyl-propane |
| (S)-eTCFP | (S)-2-{[(di-t-butyl-phosphanyl)-ethyl]-methyl-phosphanyl}- |
| | 2-methyl-propane |
| (R)-mTCFP | (R)-2-{[(di-t-butyl-phosphanyl)-methyl]-methyl- |
| | phosphanyl}-2-methyl-propane |
| (S)-mTCFP | (S)-2-{[(di-t-butyl-phosphanyl)-methyl]-methyl- |
| | phosphanyl}-2-methyl-propane |
| TEA | triethanolamine |
| Tf | triflyl or trifluoromethylsulfonyl |
| TFA | trifluoroacetic acid |
| THF | tetrahydrofuran |
| TLC | thin-layer chromatography |
| TMS | trimethylsilyl |
| Tr | trityl or triphenylmethyl |
| Ts | tosyl or p-toluenesulfonyl |

Some of the schemes and examples below may omit details of common reactions, including oxidations, reductions, and so on, separation techniques, and analytical procedures, which are known to persons of ordinary skill in the art of organic chemistry. The details of such reactions and techniques can be found in a number of treatises, including Richard Larock, Comprehensive Organic Transformations (1999), and the multi-volume series edited by Michael B. Smith and others, Compendium of Organic Synthetic Methods (1974-2005). In many cases, starting materials and reagents may be obtained from commercial sources or may be prepared using literature methods. Some of the reaction schemes may omit minor products resulting from chemical transformations (e.g., an alcohol from the hydrolysis of an ester, CO₂ from the decarboxylation of a diacid, etc.). In addition, in some instances, reaction intermediates may be used in subsequent steps without isolation or purification (i.e., in situ).

In some of the reaction schemes and examples below, certain compounds can be prepared using protecting groups, which prevent undesirable chemical reaction at otherwise reactive sites. Protecting groups may also be used to enhance solubility or otherwise modify physical properties of a compound. For a discussion of protecting group strategies, a description of materials and methods for installing and removing protecting groups, and a compilation of useful protecting groups for common functional groups, including amines, carboxylic acids, alcohols, ketones, aldehydes, and the like, see T. W. Greene and P. G. Wuts, Protecting Groups in Organic Chemistry (1999) and P. Kocienski, Protective Groups (2000), which are herein incorporated by reference in their entirety for all purposes.

Generally, the chemical transformations described throughout the specification may be carried out using substantially stoichiometric amounts of reactants, though certain reactions may benefit from using an excess of one or more of the reactants. Additionally, many of the reactions disclosed throughout the specification may be carried out at about RT and ambient pressure, but depending on reaction kinetics, yields, and the like, some reactions may be run at elevated pressures or employ higher (e.g., reflux conditions) or lower (e.g., −70° C. to 0° C.) temperatures. Many of the chemical transformations may also employ one or more compatible solvents, which may influence the reaction rate and yield. Depending on the nature of the reactants, the one or more solvents may be polar protic solvents (including water), polar aprotic solvents, non-polar solvents, or some combination. Any reference in the disclosure to a stoichiometric range, a temperature range, a pH range, etc., whether or not expressly using the word “range,” also includes the indicated endpoints.

Unless stated otherwise, when a particular substituent identifier (R¹, R², R³, etc.) is defined for the first time in connection with a formula, the same substituent identifier, when used in a subsequent formula, will have the same definition as in the earlier formula. Thus, for example, if R²⁰ in a first formula is hydrogen atom, halogeno, or C₁₋₆ alkyl, then unless stated differently or otherwise clear from the context of the specification, R²⁰ in a second formula is also hydrogen, halogeno, or C₁₋₆ alkyl.

This disclosure concerns materials and methods for preparing optically active β-amino acids represented by Formula 1, above, including opposite enantiomers thereof and diastereomers thereof and pharmaceutically acceptable complexes, salts, solvates and hydrates thereof. The claimed and disclosed methods provide compounds of Formula 1 that are stereoisomerically enriched, and which in many cases, are pure or substantially pure stereoisomers.

The compounds of Formula 1 have at least two stereogenic centers, as denoted by wedged bonds, and include substituents R¹, R² and R³, which are defined above. Compounds of Formula 1 include those in which R¹ and R² are each independently selected from hydrogen atom and C₁₋₆ alkyl, and R³ is selected from C₁₋₆ alkyl, C₃₋₆ cycloalkyl, C₃₋₆ cycloalkyl-C₁₋₃ alkyl, phenyl, phenyl-C₁₋₃ alkyl, pyridyl, and pyridyl-C₁₋₃ alkyl, wherein each alkyl or cycloalkyl moiety is optionally substituted with from one to five fluorine atoms, and each phenyl and pyridyl moiety is optionally substituted with from one to three substituents independently selected from chloro, fluoro, amino, nitro, cyano, C₁₋₃ alkylamino, C₁₋₃ alkyl optionally substituted with from one to three fluorine atoms, and C₁₋₃ alkoxy optionally substituted with from one to three fluorine atoms.

Furthermore, compounds of Formula 1 include those in which R¹ is a hydrogen atom, R² is a C₁₋₆ alkyl, including methyl, and R³ is a hydrogen atom or a C₁₋₆ alkyl, including methyl or ethyl. Compounds of Formula 1 also include those in which R¹ and R² are both C₁₋₆ alkyl, including methyl, and R³ is a hydrogen atom or a C₁₋₆ alkyl, including methyl or ethyl. Representative compounds of Formula 1 thus include (3S,5R)-3-amino-5-methyl-heptanoic acid, (3S,5R)-3-amino-5-methyl-octanoic acid, (3S,5R)-3-amino-5-methyl-nonanoic acid, (R,R,R)-3-amino-4,5-dimethyl-hexanoic acid, (R,R,R)-3-amino-4,5-dimethyl-heptanoic acid, (R,R,R)-3-amino-4,5-dimethyl-octanoic acid, (R,R,R)-3-amino-4,5-dimethyl-nonanoic acid, their opposite enantiomers, and their diastereomers.

Scheme I shows a method of preparing the optically active β-amino acids of Formula 1. The method includes reacting a chiral allyl amine (Formula 2) with a 2-alkynoate (Formula 3), in the presence of a Lewis acid and a base, to give a chiral tertiary enamine (Formula 5). The tertiary enamine (Formula 5) is subsequently reacted with ammonia in the presence of a protic solvent to provide a chiral primary enamine (Formula 6), which undergoes asymmetric hydrogenation to give the compound of Formula 9. Alternatively, the primary enamine (Formula 6) may be acylated to give a chiral enamide (Formula 8), which subsequently undergoes asymmetric hydrogenation. In either case, the hydrogenation product (Formula 9) is optionally converted to the β-amino acid (Formula 1) or to a pharmaceutically acceptable complex, salt, solvate or hydrate thereof.

As noted above, the method shown in Scheme I includes reacting a chiral allyl amine (Formula 2) with a 2-alkynoate (Formula 3) to give a chiral tertiary enamine (Formula 5). The chiral allyl amine may be prepared using methods described in the examples and includes an asymmetric α-carbon, relative to the nitrogen atom, which along with the geometric configuration of the double bond generates the desired stereochemical configuration of the enamine (Formula 5). One may also obtain an enamine (formula 5) having the same absolute stereochemical configuration by utilizing a trans chiral allyl amine having an oppositely configured stereocenter. Although Scheme I shows a stereogenic carbon attached to R³, the stereocenter may reside on an α-carbon of substituent R⁴ or R⁵.

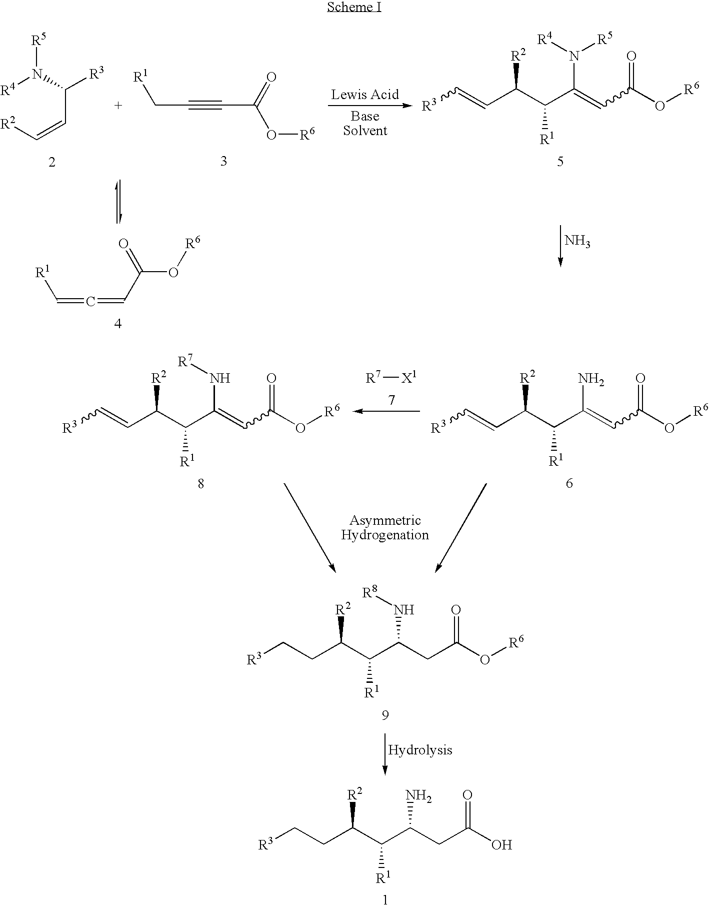

Representative chiral allyl amines (Formula 2), alkynoates (Formula 3) and chiral tertiary enamines (Formula 5) include those in which R¹ is a hydrogen atom, R² is a C₁₋₆ alkyl (e.g., methyl), and R³ is a hydrogen atom or a C₁₋₆ alkyl (e.g., methyl or ethyl), or those in which R¹ and R² are both C₁₋₆ alkyl (e.g., methyl) and R³ is a hydrogen atom or a C₁₋₆ alkyl (e.g., methyl or ethyl). Additionally or alternatively, representative chiral allyl amines, alkynoates and chiral tertiary enamines include those in which R⁴ and R⁵ are each independently methyl, ethyl, propyl or isopropyl, or those in which R⁴ and R⁵, and the nitrogen atom to which they are attached, form pyrrolidine, piperidine, or morpholine rings, including (S)— or (R)-2-methyl-pyrrolidine, and those in which R⁶ is C₁₋₆ alkyl. Representative chiral allyl amines thus include the E- and Z-isomers of (S)-1-(but-2-enyl)-2-methyl-pyrrolidine, (R)-1-(1-methyl-but-2-enyl)-pyrrolidine, (R)-1-(1-ethyl-but-2enyl)-pyrrolidine, and their opposite enantiomers. Representative alkynoates include C₁₋₆ alkyl esters of but-2-ynoic acid and pent-2-ynoic acid, such as but-2-ynoic acid ethyl ester and pent-2-ynoic acid ethyl ester. Representative chiral tertiary enamines include C₁₋₆ alkyl (e.g., Me, Et, i-Pr or n-Pr) esters of the E- and Z-isomers of (2S,5S)-5-methyl-3-(2-methyl-pyrrolidin-1-yl)-hepta-2,6-dienoic acid, (2S,4R,5R)-4,5-dimethyl-3-(2-methyl-pyrrolidin-1-yl)-hepta-2,6-dienoic acid, (S)-5-methyl-3-pyrrolidin-1-yl-octa-2,6-dienoic acid, (R,R)-4,5-dimethyl-3-pyrrolidin-1-yl-octa-2,6-dienoic acid, (S)-5-methyl-3-pyrrolidin-1-yl-nona-2,6-dienoic acid, (R,R)-4,5-dimethyl-3-pyrrolidin-1-yl-nona-2,6-dienoic acid, their opposite enantiomers, and their diastereomers.

Under the reaction conditions of this disclosure, the 2-alkynoate (Formula 3) is in dynamic equilibrium with a corresponding 3-alkynoate and a small amount (about 1% to 2%) of an alkyl 2,3-dienoate (Formula 4, in which R¹ and R⁶ are as defined above for Formula 1 and Formula 5, respectively). Though not bound to any particular theory, it appears that as the 2,3-dienoate is formed, it is attacked by the nucleophilic chiral allyl amine (Formula 2). A recent article reports that allenes may react diastereoselectively with allyl amines. See T. H. Lambert & D. W. C. MacMillan, J. Am. Chem. Soc. 124:13646-47 (2002). However, none of the allyl amines reported in Lambert et al. are chiral nor do they exhibit substitution at the α-carbon (i.e., non-hydrogen R³ in Formula 2). Furthermore, the allene esters reported in Lambert et al. are not commercially available, cannot be stored at RT without degrading, and are problematic for use in commercial-scale processes because of their potential for exothermic decomposition at moderate temperatures (DSC onset at 40° C. to 60° C.). In contrast, the 2-alkynoates Formula 3) shown in Scheme I are, in many cases, comparatively inexpensive and commercially available and, while possessing similar amount of thermal energy as the allene esters, have a relatively high exothermic onset (i.e., greater than 300° C.).

As noted above, the chiral allyl amine (Formula 2) reaction is carried out in the presence of a Lewis acid and a base. Representative bases include non-nucleophilic (hindered) bases such as Et₃N (e.g., bases whose conjugate acids have a pKa in a range of about 9 to 11). Representative Lewis acids include Group 1 or Group 2 cations obtained from an appropriate salt, such as LiBr, MgBr₂, MgCl₂, etc., and may also include compounds having the formula MXₙ, where M is Al, As, B, Fe, Ga, Mg, Nb, Sb, Sn, Ti, and Zn, X is a halogen, and n is an integer from 2 to 5, inclusive, depending on the valence state of M. Examples of compounds of formula: MXₙ include AlCl₃, AlI₃, AlF₃, AlBr₃, AsCl₃, AsI₃, AsF₃, AsBr₃, BCl₃, BBr₃, BI₃, BF₃, FeCl₃, FeBr₃, FeI₃, FeF₃, FeCl₂, FeBr₂, FeI₂, FeF₂, GaCl₃, GaI₃, GaF₃, GaBr₃, MgCl₂, MgI₂, MgF₂, MgBr₂, NbCl₅, SbCl₃, SbI₃, SbF₃, SbBr₃, SbCl₅, SbI₅, SbF₅, SbBr₅, SnCl₂, SnI₂, SnF₂, SnBr₂, SnCl₄, SnI₄, SnF₄, SnBr₄, TiBr₄, TiCl₂, TiCl₃, TiCl₄, TiF₃, TiF₄, TiI₄, ZnCl₂, ZnI₂, ZnF₂, and ZnBr₂.

Other Lewis acids, include Al₂O₃, BF₃BCl₃.SMe₂, BI₃.SMe₂, BF₃.SMe₂, BBr₃.SMe₂, BF₃.OEt₂, Et₂AlCl, EtAlCl₂, MgCl₂.OEt₂, MgI₂.OEt₂, MgF₂.OEt₂, MgBr₂.OEt₂, Et₂AlCl, EtAlCl₂, LiClO₄, Ti(O-i-Pr)₄, and Zn(OAc)₂. Still other Lewis acids include salts of cobalt (II), copper (II), and nickel (II), such as (CH₃CO₂)₂Co, CoBr₂, CoCl₂, CoF₂, CoI₂, Co(NO₃)₂, cobalt (II) triflate, cobalt (II) tosylate, (CH₃CO₂)₂Cu, CuBr₂, CuCl₂, CuF₂, CuI₂, Cu(NO₃)₂, copper (II) triflate, copper (II) tosylate, (CH₃CO₂)₂Ni, NiBr₂, NiCl₂, NiF₂, NiI₂, Ni(NO₃)₂, nickel (II) triflate, and nickel (II) tosylate. Monoalkyl boronhalides, dialkyl boronhalides, monoaryl boronhalides, and diaryl boronhalides may be employed as Lewis acids. In addition, rare earth metal trifluoromethansulfonates such as Eu(OTf)₃, Dy(OTf)₃, Ho(OTf)₃, Er(OTf)₃, Lu(OTf)₃, Yb(OTf)₃, Nd(OTf)₃, Gd(OTf)₃, Lu(OTf)₃, La(OTf)₃, Pr(OTf)₃, Tm(OTf)₃, Sc(OTf)₃, Sm(OTf)₃, AgOTf, Y(OTf)₃, and polymer resins thereof (e.g., scandium triflate polystyrene resin, PS—Sc(OTf)₂) may be used in a solution such as one pall water and four to nine parts THF. Other Lewis acids may include, silica gels such as silica gel (CAS 112926-00-8) used for column chromatography (80-500 mesh particle size).

The reaction typically employs stoichiometric amounts of the chiral allyl amine Formula 2) and 2-alkynoate (Formula 3) though the reaction may benefit from excess 2-alkynoate and base (e.g., about 1.1 eq to about 1.5 eq). The Lewis acid may be used in catalytic amounts (e.g., from about 5 mol % to about 10 mol %), but may be used in higher amounts as well (e.g., from about 1 eq to about 1.5 eq). Likewise, the base may be employed in stoichiometric amounts or in slight excess (e.g., from about 1.1 eq to about 1.5 eq) relative to the limiting reactant. The reaction may be carried out in a compatible solvent at a temperature of about RT to about 90° C., or more typically, at a temperature of about 40° C. to about 90° C. Typical solvents include polar aprotic solvents such as ACN, DMF, DMSO, MeCl₂, and the like.

As shown in Scheme I, the chiral tertiary enamine (Formula 5) is converted to a chiral primary enamine (Formula 6) via reaction with ammonia in the presence a protic solvent. Representative solvents include alkanols, such as MeOH, EtOH, n-Pr, i-Pr, and the like, as well as mixtures of water and a polar aprotic solvent, such as ACN, DMF, DMSO, and the like. The ammonia exchange reaction is carried out at a temperature that may range from about RT to reflux and commonly ranges from about 40° C. to about 60° C. The reaction generally employs a large excess of ammonia (e.g., 10 eq or more) in which the NH₃ concentration in the solvent lies in a range of about 1.5 M to about 3.0 M.

As shown in Scheme I, the method also provides fur optionally converting the chiral primary enamine (Formula 6) to the enamide (Formula 8) via contact with an acylating agent (Formula 7). Representative optically active primary enamines (Formula 6) include C₁₋₆ alkyl (e.g., Me, Et, i-Pr or n-Pr) esters of the E- and Z-isomers of (S)-3-amino-5-methyl-hepta-2,6-dienoic acid, (S)-3-amino-5-methyl-octa-2,6-dienoic acid, (S)-3-amino-5-methyl-nona-2,6-dienoic acid, (R,R)-3-amino-4,5-dimethyl-hepta-2,6-dienoic acid, (R,R)-3-amino-4,5-dimethyl-octa-2,6-dienoic acid, (R,R)-3-amino-4,5-dimethyl-nona-2,6-dienoic acid, their opposite enantiomers, and their diastereomers. Useful acylating agents include carboxylic acids, which have been activated either prior to contacting the enamine (Formula 6) or in-situ (i.e., in the presence of the enamine using an appropriate coupling agent). Representative activated carboxylic acids (Formula 7) include acid halides, anhydrides, mixed carbonates, and the like, in which X¹ is a leaving group, such as halogeno, aryloxy (e.g. phenoxy, 3,5-dimethoxyphenoxy, etc.) and heteroaryloxy (e.g., imidazolyloxy), or —OC(O)R⁹, in which R⁹ is C₁₋₆ alkyl, C₂₋₆ alkenyl, C₂₋₆ alkynyl, C₃₋₁₂ cycloalkyl, halo-C₁₋₆ alkyl, halo-C₂₋₆ alkenyl, halo-C₂₋₆ alkynyl, aryl, aryl-C₁₋₆ alkyl, heterocyclyl, heteroaryl, or heteroaryl-C₁₋₆ alkyl.

Other suitable acylating agents may include carboxylic acids, which ate activated in-situ using a coupling agent. Typically, the reaction is carried out in an aprotic solvent, such as ACN, DMF, DMSO, toluene, MeCl₂, NMP, THF, and the like, and may also employ a catalyst. Coupling agents include DCC, DMT-MM, FDPP, TATU, BOP, PyBOP, EDCI, diisopropyl carbodiimide, isopropenyl chloroformate, isobutyl chloroformate, N,N-bis-(2-oxo-3-oxazolidinyl)-phosphinic chloride, diphenylphosphoryl azide, diphenylphosphinic chloride, and diphenylphosphoryl cyanide. Catalysts for the coupling reaction may include DMAP, HODhbt, HOBt, and HOAt.

The optically active primary enamine (Formula 6) or enamide (Formula 8) undergoes asymmetric hydrogenation in the presence of a catalyst to give the compound of Formula 9. As depicted in Scheme I, representative enamide hydrogenation substrates (Formula 8) include individual Z- or E-isomers or a mixture of Z- and E-isomers, and include C₁₋₆ alkyl (e.g., Me, Et, i-Pr or n-Pr) esters of the Z- and E-isomers of (S)-3-acetylamino-5-methyl-hepta-2,6-dienoic acid, (S)-3-acetylamino-5-methyl-octa-2,6-dienoic acid, (S)-3-acetylamino-5-methyl-nona-2,6-dienoic acid, (R,R)-3-acetylamino-4,5-dimethyl-hepta-2,6-dienoic acid, (R,R)-3-acetylamino-4,5-dimethyl-octa-2,6-dienoic acid, (R,R)-3-acetylamino-4,5-dimethyl-nona-2,6-dienoic acid, their opposite enantiomers, and their diastereomers.

When substituent R⁶ in Formula 6 or Formula 8 is a hydrogen atom, the method may optionally include converting the carboxylic acid to a Group 1, Group 2, or ammonium salt prior to asymmetric hydrogenation through contact with a suitable base, such as a primary amine (e.g., t-BuNH₂), a secondary amine (DIPEA), and the like. In some instances, the use of a salt of the enamine (Formula 6) or enamide (Formula 8) may increase conversion, improve stereoselectivity, or provide other advantages. Optionally, the method may employ an inorganic salt of the carboxylic acid obtained through contact with a suitable base such as NaOH, Na₂CO₂, LiOH, Ca(OH)₂, and the like.

Depending on which enantiomer or diastereomer of the chiral catalyst is used, the asymmetric hydrogenation generates an excess (de) of a diastereoisomer of Formula 9. Although the amount of the desired diastereoisomer produced will depend on, among other things, the choice of chiral catalyst, a de of the desired diastereoisomer of about 50% or greater is desirable; a de of about 70% or greater is more desirable; and a de of about 85% is still more desirable. Particularly useful asymmetric hydrogenations are those in which the de of the desired diastereoisomer is about 90% or greater. For the purposes of this disclosure, a desired diastereoisomer or enantiomer is considered to be substantially pure if it has a de or ee of about 90% or greater.

Generally, the asymmetric hydrogenation of the enamine (Formula 6) or enamide (Formula 8) employs a chiral catalyst having the requisite stereochemistry. Useful chiral catalysts include cyclic or acyclic, chiral phosphine ligands (e.g., monophosphines, bisphosphines, bisphospholanes, etc.) or phosphinite ligands bound to transition metals, such as ruthenium, rhodium, iridium or palladium. Ru—, Rh—, Ir—or Pd-phosphine, phosphinite or phosphino oxazoline complexes are optically active because they possess a chiral phosphorus atom or a chiral group connected to a phosphorus atom, or because in the case of BINAP and similar atropisomeric ligands, they possess axial chirality. Useful chiral ligands include BisP*; (R)-BINAPINE; (S)-Me-ferrocene-Ketalphos, (R,R)-DIOP; (R,R)-DIPAMP; (R)—(S)-BPPFA; (S,S)-BPPM; (+)-CAMP; (S,S)-CHIRAPHOS; (R)-PROPHOS; (R,R)-NORPHOS; (R)-BINAP; (R)-CYCPHOS; (R,R)-BDPP; (R,R)-DEGUPHOS; (R,R)-Me-DUPHOS; (R,R)-Et-DUPHOS; (R,R)-i-Pr-DUPHOS; (R,R)-Me-BPE; (R,R)-Et-BPE (R)-PNNP; (R)-BICHEP; (R,S,R,S)-Me-PENNPHOS; (S,S)-BICP; (R,R)-Et-FerroTANE; (R,R)-t-butyl-miniPHOS; (R)-Tol-BINAP; (R)-MOP; (R)-QUINAP; CARBOPHOS; (R)—(S)-JOSIPHOS; (R)-PHANEPHOS; BIPHEP; (R)—Cl-MeO-BIPHEP; (R)-MeO-BIPHEP; (R)-MonoPhos; BIFUP; (R)-SpirOP; (+)-TMBTP; (+)-tetraMeBITIANP; (R,R,S,S) TANGPhos; (R)-PPh₂-PhOx-Ph; (S,S) MandyPhos; (R)-eTCFP; (R)-mTCFP; and (R)-CnTunaPHOS, where n is an integer of 1 to 6.

Other useful chiral ligands include (R)-(−)-1-[(S)-2-(di(3,5-bistrifluoromethylphenyl)phosphino)ferrocenyl]ethyldicyclohexyl-phosphine; (R)-(−)-1-[(S)-2-(di(3,5-bis-trifluoromethylphenyl)phosphino)ferrocenyl]ethyldi(3,5-dimethylphenyl)phosphine; (R)-(−)-1-[(S)-2-(di-t-butylphosphino)ferrocenyl]ethyldi(3,5-dimethylphenyl)phosphine; (R)-(−)-1-[(S)-2-(dicyclohexylphosphino)ferrocenyl]ethyldi-t-butylphosphine; (R)-(−)-1-[(S)-2-(dicyclohexylphosphiino)ferrocenyl]ethyldicyclohexylphosphine; (R)-(−)-1-[(S)-2-(dicyclohexylphosphino)ferrocenyl]ethyldiphenylphosphine; (R)-(−)-1-[(S)-2-(di(3,5-dimethyl-4-methoxyphenyl)phosphino)ferrocenyl]ethyldicyclohexylphosphine; (R)-(−)-1-[(S)-2-(diphenylphosphino)ferrocenyl]ethyldi-t-butylphosphine; (R)—N-[2-(N,N-dimethylamino)ethyl]-N-methyl-1-[(S)-1′,2-bis(diphenylphosphino)ferrocenyl]ethylamine; (R)-(+)-2-[2-(diphenylphosphino)phenyl]-4-(1-methylethyl)-4,5-dihydrooxazole; {1-[((R,R)-2-benzyl-phospholanyl)-phen-2-yl]-(R*,R*)-phospholan-2-yl}-phenyl-methane; and {1-[((R,R)-2-benzyl-phospholanyl)-ethyl]-(R*,R*)-phospholan-2-yl}-phenyl-methane.

Useful ligands may also include stereoisomers (enantiomers and diastereoisomers) of the chiral ligands described in the preceding paragraphs, which may be obtained by inverting all or some of the stereogenic centers of a given ligand or by inverting the stereogenic axis of an atropoisomeric ligand. Thus, for example, useful chiral ligands may also include (S)—Cl-MeO-BIPHEP; (S)-PHANEPHOS; (S,S)-Me-DUPHOS; (S,S)-Et-DUPHOS; (S)-BINAP; (S)-Tol-BINAP; (R)—(R)-JOSIPHOS; (S)—(S)-JOSIPHOS; (S)-eTCFP; (S)-mTCFP and so on.

Many of the chiral catalysts, catalyst precursors, or chiral ligands may be obtained from commercial sources or may be prepared using known methods. A catalyst precursor or pre-catalyst is a compound or set of compounds, which are converted into the chiral catalyst prior to use. Catalyst precursors typically comprise Ru, Rh, Ir or Pd complexed with the phosphine ligand and either a diene (e.g., NBD, COD, (2-methylallyl)₂, etc.) or a halide (Cl or Br) or a diene and a halide, in the presence of a counterion, X⁻, such as OTf⁻, PF₆⁻, BF₄⁻, SbF₆⁻, ClO₄⁻, etc. Thus, for example, a catalyst precursor comprised of the complex, [(bisphosphine ligand)Rh(COD)]⁺X⁻ may be converted to a chiral catalyst by hydrogenating the diene (COD) in MeOH to yield [(bisphosphine ligand)Rh(MeOH)₂]⁺X⁻. MeOH is subsequently displaced by the enamine (Formula 6) or the enamide (Formula 8), which undergoes enantioselective hydrogenation to the desired chiral compound (Formula 9). Examples of chiral catalysts or catalyst precursors include (+)-TMBTP-ruthenium(II) chloride acetone complex; (S)—Cl-MeO-BIPHEP-ruthenium(II) chloride Et₃N complex; (S)-BINAP-ruthenium(II) Br₂ complex; (S)-tol-BINAP-ruthenium(II) Br₂ complex; [((3R,4R)-3,4-bis(diphenylphosphino)-1-methylpyrrolidine)-rhodium-COD]-tetrafluoroborate complex; [((R,R,S,S)-TANGPhos)-rhodium(I)-bis(COD)]-trifluoromethane sulfonate complex; [(R)-BINAPINE-rhodium-COD]-tetrafluoroborate complex; [(S)-eTCFP-COD-rhodium(I)]-tetrafluoroborate complex; and [(S)-mTCFP-COD-rhodium(I)]-tetrafluoroborate complex.

For a given chiral catalyst and hydrogenation substrate (Formula 6 or 8), the molar ratio of the substrate and catalyst (s/c) may depend on, among other things, H₂ pressure, reaction temperature, and solvent (if any). Usually, the substrate-to-catalyst ratio exceeds about 100:1 or 200:1, and substrate-to-catalyst ratios of about 1000:1 or 2000:1 are common. Although the chiral catalyst may be recycled, higher substrate-to-catalyst ratios are more useful. For example, substrate-to-catalyst ratios of about 1000:1, 10,000:1, and 20,000:1, or greater, would be useful. The asymmetric hydrogenation is typically carried out at about RT or above, and under about 10 kPa (0.1 atm) or more of H₂. The temperature of the reaction mixture may range from about 20° C. to about 80° C., and the H₂ pressure may range from about 10 kPa to about 5000 kPa or higher, but more typically, ranges from about 10 kPa to about 100 kPa. The combination of temperature, H₂ pressure, and substrate-to-catalyst ratio is generally selected to provide substantially complete conversion (i.e., about 95 wt %) of the substrate (Formula 6 or 8) within about 24 h. With many of the chiral catalysts, decreasing the H₂ pressure increases the enantioselectivity.

A variety of organic solvents may be used in the asymmetric hydrogenation, including protic solvents, such as MeOH, EtOH, and i-PrOH. Other solvents may include aprotic polar solvents, such as THF, ethyl acetate, and acetone. The stereoselective hydrogenation may employ a single solvent, or may employ a mixture of solvents, such as MeOH and THF.

In some cases it may be advantageous to employ more than one chiral catalyst to carryout the asymmetric hydrogenation of the substrate (Formula 6 or 8). For example, the method may provide for reacting the enamide successively with first and second chiral catalysts to exploit the comparatively greater stereoselectivity, but lower reaction rate of the first (or second) chiral catalyst. Thus, for example, the method provides for reacting the enamide with hydrogen in the presence of a chiral. catalyst comprised of (R)-BINAPINE or its opposite enantiomer, followed by reaction in the presence of a chiral catalyst comprised of (R)-mTCFP or its opposite enantiomer.

When substituents R¹ and R² are both non-hydrogen, the enamide (Formula 8) may under asymmetric hydrogenation using an achiral catalyst. Useful catalysts include heterogeneous catalysts containing from about 0.1% to about 20%, and more typically, from about 1% to about 5%, by weight, of transition metals such as Ni, Pd, Pt, Rh, Re, Ru, and Ir, including oxides and combinations thereof, which are typically supported on various materials, including Al₂O₃, C, CaCO₃, SrCO₃, BaSO₄, MgO, SiO₂, TiO₂, ZrO₂, and the like. Many of these metals, including Pd, may be doped with an amine, sulfide, or a second metal, such as Pb, Cu, or Zn. Useful catalysts thus include palladium catalysts such as Pd/C, Pd/SrCO₃, Pd/Al₂O₃, Pd/MgO, Pd/CaCO₃, Pd/BaSO₄, PdO, Pd black, PdCl₂, and the like, containing from about 1% to about 5% Pd, based on weight. Other useful catalysts include Raney nickel, Rh/C, Ru/C, Re/C, PtO₂, Rh/C, RuO₂, and the like. For a discussion of other useful heterogeneous catalysts, see U.S. Pat. No. 6,624,112 to Hasegawa et al., which is herein incorporated by reference.

As shown in Scheme I, the method optionally provides for conversion of the hydrogenation product (Formula 9) into the optically active β-amino acid (Formula 1). For example, when R³ is C₁₋₆ alkyl and R⁸ is non-hydrogen, the ester and amide moieties may be hydrolyzed by treatment with an acid or a base or by treatment with a base (or acid) followed by treatment with an acid (or base). For example, treating the compound of Formula 9 with HCl, H₂SO₄, and the like, with excess H₂O generates the β-amino acid (Formula 1) or an acid addition salt. Treating the compound of Formula 9 with an aqueous inorganic base, such as LiOH, KOH, NaOH, CsOH, Na₂CO₃, K₂CO₃, Cs₂CO₃, and the like, in an optional polar solvent (e.g., THF, MeOH, EtOH, acetone, ACN, etc.) gives a base addition salt of a β-amido acid, which may be treated with an acid to generate the β-amino acid (Formula 1) or an acid addition salt. Likewise, when R⁸ in Formula 9 is a hydrogen atom, the ester moiety may be hydrolyzed by treatment with an acid or base to give the β-amino acid (Formula 1) or an acid or base addition salt. The ester and amide hydrolysis may be carried out at RT or at temperatures up to reflux temperature, and if desired, treatment of the acid or base addition salts with a suitable base (e.g., NaOH) or acid (e.g., HCl) gives the free amino acid (zwitterion).

Compounds represented by Formula 9 include β-amino and β-amido C₁₋₆ alkyl esters in which R¹ is a hydrogen atom, R² is a C₁₋₆ alkyl (e.g., methyl), and R³ is a hydrogen atom or a C₁₋₆ alkyl (e.g., methyl or ethyl), or those in which R¹ and R² are both C₁₋₆ alkyl (e.g., methyl) and R³ is a hydrogen atom or a C₁₋₆ alkyl (e.g., methyl or ethyl). Compounds of Formula 9 include C₁₋₆ alkyl (e.g., Me, Et, i-Pr or n-Pr) esters of (3S,5R)-3-amino-5-methyl-heptanoic acid, (3S,5R)-3-amino-5-methyl-octanoic acid, (3S,5R)-3-amino-5-methyl-nonanoic acid, (3S,5R)-3-acetylamino-5-methyl-heptanoic acid, (3S,5R)-3-acetylamino-5-methyl-octanoic acid, (3S,5R)-3-acetylamino-5-methyl-nonanoic acid, their opposite enantiomers, and their diastereomers. Other compounds of Formula 9 include C₁₋₆ alkyl esters (e.g., Me, Et, i-Pr or n-Pr) of (R,R,R)-3-amino-4,5-dimethyl-heptanoic acid, (R,R,R)-3-amino-4,5-dimethyl-octanoic acid, (R,R,R)-3-amino-4,5-dimethyl-nonanoic acid, (R,R,R)-3-acetylamino-4,5-dimethyl-heptanoic acid, (R,R,R)-3-acetylamino-4,5-dimethyl-octanoic acid, (R,R,R)-3-acetylamino-4,5-dimethyl-nonanoic acid, their opposite enantiomers, and their diastereomers.

Compounds of Formula 9 also include β-amido acids in which R¹ is a hydrogen atom, R² is a C₁₋₆ alkyl (e.g., methyl), and R³ is a hydrogen atom or a C₁₋₆ alkyl (e.g., methyl or ethyl), or those in which R¹ and R² are both C₁₋₆ alkyl (e.g., methyl) and R³ is a hydrogen atom or a C₁₋₆ alkyl (e.g., methyl or ethyl). Compounds of Formula 9 thus include (3S,5R)-3-acetylamino-5-methyl-heptanoic acid, (3S,5R)-3-acetylamino-5-methyl-octanoic acid, and (3S,5R)-3-acetylamino-5-methyl-nonanoic acid, (R,R,R)-3-acetylamino-4,5-dimethyl-heptanoic acid, (R,R,R)-3-acetylamino-4,5-dimethyl-octanoic acid, (R,R,R)-3-acetylamino-4,5-dimethyl-nonanoic acid, their opposite enantiomers, and their diastereomers.

The compounds of Formula 1, their opposite enantiomers, or their diastereoisomers, may be further enriched through, e.g., fractional recrystallization or chromatography or by recrystallization in a suitable solvent. In addition, compounds of Formula 1 or Formula 9 may be enriched through treatment with an enzyme such as a lipase or amidase.

Desired enantiomers of any of the compounds disclosed herein may be enriched through classical resolution, chiral chromatography, or recrystallization. For example, a racemic mixture of enantiomers may be reacted with an enantiomerically-pure compound (e.g., acid or base) to yield a pair of diastereoisomers, each composed of a single enantiomer, which are separated via, say, fractional recrystallization or chromatography. The desired enantiomer is subsequently regenerated from the appropriate diastereoisomer. Additionally, the desired enantiomer often may be further enriched by recrystallization in a suitable solvent when it is it available in sufficient quantity (e.g., typically not much less than about 85% ee, and in some cases, not much less than about 90% ee).

Many of the compounds described herein are capable of forming pharmaceutically acceptable salts. These salts include acid addition salts (including di-acids) and base salts. Pharmaceutically acceptable acid addition salts include nontoxic salts derived from inorganic acids such as hydrochloric, nitric, phosphoric, sulfuric, hydrobromic, hydriodic, hydrofluoric, phosphorous, and the like, as well nontoxic salts derived from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, trifluoroacetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesufonate, phenylacetate, citrate, lactate, malate, tartrate, methanesulfonate, and the like.

Pharmaceutically acceptable base salts include nontoxic salts derived from bases, including metal cations, such as an alkali or alkaline earth metal cation, as well as amines. Examples of suitable metal cations include sodium cations (Na⁺), potassium cations (K⁺), magnesium cations (Mg²⁺), calcium cations (Ca²⁺), and the like. Examples of suitable amines include N,N′-dibenzylethylenediamine, chloroprocaine, choline, diethanolamine, dicyclohexylamine, ethylenediamine, N-methylglucamine, and procaine. For a discussion of useful acid addition and base salts, see S. M. Berge et al., “Pharmaceutical Salts,” 66 J. of Pharm. Sci., 1-19 (1977); see also Stahl and Wermuth, Handbook of Pharmaceutical Salts: Properties, Selection, and Use (2002).

One may prepare an acid addition salt (or base salt) by contacting a compound's free base (or free acid) with a sufficient amount of a desired acid (or base) to produce a nontoxic salt. One may then isolate the salt by filtration if it precipitates from solution, or by evaporation to recover the salt. One may also regenerate the free base (or free acid) by contacting the acid addition salt with a base (or the base salt with an acid). Certain physical properties (e.g., solubility, crystal structure, hygroscopicity, etc.) of a compound's free base, free acid, or zwitterion may differ from its acid or base addition salt. Generally, however, references to the free acid, free base or zwitterion of a compound would include its acid and base addition salts.

Disclosed and claimed compounds may exist in both unsolvated and solvated forms and as other types of complexes besides salts. Useful complexes include clathrates or compound-host inclusion complexes where the compound and host are present in stoichiometric or non-stoichiometric amounts. Useful complexes may also contain two or more organic, inorganic, or organic and inorganic components in stoichiometric or non-stoichiometric amounts. The resulting complexes may be ionized, partially ionized, or non-ionized. For a review of such complexes, see J. K. Haleblian, J. Pharm. Sci. 64(8):1269-88 (1975). Pharmaceutically acceptable solvates also include hydrates and solvates in which the crystallization solvent may be isotopically substituted, e.g. D₂O, d₆-acetone, d₆-DMSO, etc. Generally, for the purposes of this disclosure, references to an unsolvated form of a compound also include the corresponding solvated or hydrated form of the compound.

The disclosed compounds also include all pharmaceutically acceptable isotopic variations, in which at least one atom is replaced by an atom having the same atomic number, but an atomic mass different from the atomic mass usually found in nature. Examples of isotopes suitable for inclusion in the disclosed compounds include isotopes of hydrogen, such as ²H and ³H; isotopes of carbon, such as ¹³C and ¹⁴C; isotopes of nitrogen, such as ¹⁵N; isotopes of oxygen, such as ¹⁷O and ¹⁸O; isotopes of phosphorus, such as ³¹P and ³²P; isotopes of sulfur, such as ³⁵S; isotopes of fluorine, such as ¹⁸F; and isotopes of chlorine, such as ³⁶Cl. Use of isotopic variations (e.g., deuterium, ²H) may afford certain therapeutic advantages resulting from greater metabolic stability, for example, increased in vivo half-life or reduced dosage requirements. Additionally, certain isotopic variations of the disclosed compounds may incorporate a radioactive isotope (e.g., tritium, ³H, or ¹⁴C), which may be useful in drug and/or substrate tissue distribution studies.

### EXAMPLES

The following examples are intended as illustrative and non-limiting, and represent specific embodiments of the present invention.

### Example 1

### Preparation of (±)-2-pyrrolidin-1-yl-propionitrile

Aqueous HCl (37 wt %, 84.4 g, 851 mmol, 1.02 eq) was added to a solution of pyrrolidine (59.9 g. 843 mmol) and water (400 mL) having an initial temperature of 17° C. During the addition of the acid, the mixture was maintained at a temperature less than 23° C. The mixture was subsequently cooled to −2° C. and KCN (56.3 g, 865 mmol, 1.03 eq) was added. The mixture was warmed to 4° C. and the resulting solution was added to a mixture of acetaldehyde (37.5 g, 852 mmol, 1.01 eq) and MTBE (263 g) while maintaining the mixture at a temperature less than 16° C. Water (37 g) was added to the mixture, which was stirred at RT for 16 h, and the resulting organic and aqueous phases were separated. The organic fraction was washed with saturated aq NaCl (50 mL), and the aqueous fraction was extracted with MTBE (100 mL). The organic fractions were combined and dried over MgSO₄ and concentrated to give (±)-2-pyrrolidin-1-yl-propionitrile as an oil (96.6 g, 92%). ¹H NMR (400 MHz, CDCl₃) δ 1.49 (d, J=7 Hz, 3 H), 1.85 (m, 4 H), 2.64 (m, 2 H), 3.89 (q, J=7 Hz, 1 H);¹³C NMR (CDCl₃) δ 18.70, 23.37, 49.75, 49.86, 118.00; MS (ESI+) for C₇H₁₂N₂ m/z 125 (M+H, 100); GC t_{R}=2.94 min, column: DB-1, 15 m×0.25 mm ID×0.25 μm film thickness, oven: Tᵢₙᵢ=90° C., ramp to 310° C. at 7° C./min, Tᵢₙⱼ=230° C., T_{det}=250° C., sample preparation: 10 mg/mL in MeOH.

### Example 2

### Preparation of (Z)-(propenylmagnesium)bromide

(Z)-(Propenylmagnesium)bromide in THF (0.53 M, 14.7 mL, 7.79 mmol, 0.011 eq) was added to a suspension of magnesium (17.63 g, 725 mmol, 1.17 eq) in THF (350 mL) and 1,10-phenanthroline monohydrate (0.106 g, 0.53 mmol, 0.00086 eq) to a persistent purple endpoint. During the addition, the mixture was maintained at a temperature of 20-25° C. (Note: for the initial preparation, commercial methylmagnesium bromide in THF can be substituted). Over a 2 h period, (Z)-1-bromo-propene (74.8 g. 618.3 mmol) was added to the mixture via a syringe pump with a THF rinse (567 mL) while maintaining the mixture at a temperature of 20-25° C. The mixture was stirred at RT for 16 h. A sample of the resulting purple solution was titrated to a pink end point with s-butanol in xylenes, which indicated that the solution contained (Z)-(propenylmagnesium)bromide at a concentration of 0.545 M. The total volume of supernatant was 870 mL (474 mmol, 76.7%).

### Example 3

### Preparation of (±)-(Z)-1-(1-methyl-but-2-enyl)-pyrrolidine

A solution of (Z-propenylmagnesium)bromide in THF (0.545 M, 740 mL, 403 mmol, 1.11 eq) was added to a −10° C. solution of (±)-2-pyrrolidin-1-yl-propionitrile (45.0 g, 362.6 mmol) in THF (100 mL) while maintaining the temperature of the mixture at less than 14° C. The mixture was stirred at 22-23° C. for 1 h. Water (250 mL) was subsequently added, followed by MTBE (250 mL) and acetic acid (35.95 g, 599 mmol, 1.65 eq) while maintaining the mixture at a temperature less than 26° C. The resulting aqueous and organic phases were separated. The organic fraction was washed with sodium bicarbonate (25.95 g) in water (251 g), and the aqueous fraction was extracted with MTBE (250 mL). The organic fractions were combined and washed with saturated aq NaCl (50 mL) and the brine was back extracted with MTBE (100 mL). The combined organic extracts were dried over MgSO₄ and concentrated to give a crude oil. The sequence was repeated with 41.4 g of (±)-2-pyrrolidin-1-yl-propionitrile. The combined crude oils were purified by vacuum distillation (bp 52-64° C. at 7 Torr) to afford (±)-(Z)-1-(1-methyl-but-2-enyl)-pyrrolidine as a colorless oil (47.29 g, 44%). ¹H NMR (400 MHz, CDCl₃) δ 1.16 (d, J=8 Hz, 3 H), 1.64 (d, J=6 Hz, 3 H), 1.78 (m, 4 H), 2.51 (m, 4 H),3.10 (m, 1 H), 5.44 (m, 2 H); ¹³C NMR (CDCl₃) δ 13.18, 20.64, 23.32, 52.01, 56.33, 123.53, 134.33; MS (ESI+) for C₉H₁₇N m/z 140 (M+H, 100); GC t_{R}=2.78 min, column: DB-1, 15 m×0.25 mm ID×0.25 μm film thickness, oven: Tᵢₙᵢ=90° C., ramp to 310° C. at 7° C./ min, Tᵢₙⱼ=230° C., T_{det}=250° C., sample preparation: 10 mg/mL in MeOH.

### Example 4

### Preparation of 1-[(1R,2Z)-1-methyl-but-2-en-1-yl]-pyrrolidine, di-p-toluoyl-L-tartaric acid salt (1:1)

(±)-(Z)-1-(1-Methyl-but-2-enyl)-pyrrolidine (33.58 g, 241 mmol) was added to a solution of di-p-toluoyl-L-tartaric acid (90.18 g, 233 mmol, 0.968 eq) in MeOH (449 g) which yielded a white slurry. Toluene (508 g) was added and the mixture was stirred at 24° C. for 20 min. The product was collected by vacuum filtration, washed with toluene, and dried in a nitrogen stream to give a crude salt (36.96 g, 80% ee by chiral GC). The procedure was repeated to afford additional crude salt. MeOH (1 kg) was added to the crude salt (44.06 g) and the resulting slurry was warmed to 62° C. to afford a solution. The solution was cooled to 34° C. to form a slurry, which was concentrated in vacuo (637 g). Toluene (635 g) was added and the resulting precipitate was collected by vacuum filtration, washed with toluene, and dried in a nitrogen stream to afford 1-[(1R,2Z)-1-methyl-but-2-en-1-yl]-pyrrolidine, di-p-toluoyl-L-tartaric acid salt (24.45 g, 56% recovery, 98.0% ee by GC); GC t_{R}=19.65 min, column: Beta CD 120, 30 m×0.25 mm ID×0.25 μm film thickness by Supelco, oven: 70° C. for 15 min, ramp to 220° C. at 20° C./min, hold for 5 min at 220° C., Tᵢₙⱼ=230° C., T_{det}=250° C., sample preparation: 10mg/mL in MTBE (0.5 mL) and 1M NaOH (0.5 mL), inject upper phase; ¹H NMR (400 MHz, 1:1 DMSO-d₆: MeOH-d₄) δ 2.01 (d, J=7 Hz, 3 H), 2.40 (dd, J=2, 7 Hz, 3 H), 2.63 (m, 4 H), 3.12 (s, 6 H), 4.85 (p, J=7 Hz, 1 H), 5.15 (s, 6 H), 6.14 (t, J=10 Hz, 1 H), 6.47 (m, 3 H), 8.05 (d, J=8 Hz, 4 H), 8.70 (d, J=8 Hz, 4 H); ¹³C No (DMSO-d₆: MeOH-d₄) δ 15.08, 19.99, 23.31, 25.40, 53.58, 59.49, 76.09, 128.28, 129.80, 131.70, 132.33, 133.49, 146.72, 168.20, 172.06; MS (ESI+) for C₉H₁₇N m/z 140 (M+H, 100); MS (ESI−) for C₂₀H₁₈O₈ m/z 385 (M−H, 6), 135 (48), 113 (100); [α]²²_{D} (−93.99, C=1.0, 1:1 DMSO: MeOH); Anal. Calc'd for C₉H₁₇N.C₂₀H₁₈O₈: C, 66.27; H, 6.71; N, 2.66. Found: C, 66.27; H, 6.69; N, 2.64.

### Example 5

### Preparation of 1-[(1R,2Z)-1-methyl-but-2-en-1-yl]-pyrrolidine

Water (161 g) and MeCl₂ (95.6 g) were added to 1-[(1R,2Z)-1-methyl-but-2-en-1-yl]-pyrrolidine, di-p-toluoyI-L-tartaric acid salt (1:1) (25.55 g, 48.6 mmol). The pH was adjusted to 12.6 with NaOH aq (50%, 9.14 g, 114 mmol, 2.35 eq) and the resulting aqueous and organic phases were separated. The aqueous fraction was washed with MeCl₂ (70 g). The organic extracts were combined, dried over MgSO₄, and concentrated to a colorless oil. Pentane was added, and the solution was concentrated to give 1-[(1R,2Z)-1-methyl-but-2-en-1-yl]-pyrrolidine as a colorless oil (6.94 g, 102%). ¹H NMR (400 MHz, CDCl₃) δ 1.16 (d, J=8 Hz, 3 H), 1.64 (d, J=6 Hz, 3 H), 1.78 (m, 4 H), 2.51 (m, 4 H), 3.10 (m, 1 H), 5.44 (m, 2 H); ³C NMR (CDCl₃) δ 13.18, 20.64, 23.32, 52.01, 56.33, 123.53, 134.33; MS (ESI+) for C₉H₁₇N m/z 140 (M+H, 100); [α]²²_{D} (20.51, C=1.0, CH₂Cl₂); Anal. Calc'd for C₉H₁₇N: C, 77.63; H, 12.31; N, 10.06. Found: C, 77.48; H, 12.48; N, 9.93.

### Example 6

### Preparation of (S)-methanesulfonic acid 1-methyl-but-2-ynyl ester

Methanesulfonyl chloride (3.28 mL, 42.4 mmol, 1.18 eq) was added to a solution of (S)-3-pentyn-2-ol (3.03 g, 36.0 mmol) in MeCl₂ and Et₃N (8.70 mL, 62.4 mmol, 1.73 eq), which was initially at a temperature of 4° C. During the addition of MsCl, the temperature of the solution was maintained at a temperature less than 11° C. The resulting slurry was stirred at 8° C. for 1 h. Aqueous HCl was added to an aliquot of the reaction mixture; the resulting phases were separated and the organic fraction was dried over MgSO₄ and concentrated in vacuo to afford (S)-methanesulfonic acid 1-methyl-but-2-ynyl ester. ¹H NMR (400 MHz, CDCl₃) δ 1.61 (d, J=7 Hz, 3 H), 1.89 (d, J=2 Hz, 3 H), 3.11 (s, 3 H), 5.27 (m, 1 H); ¹³C N NMR (CDCl₃) δ 3.54, 22.87, 39.04, 68.90, 75.96, 84.89; GC t_{R}=4.65 min, column: DB-1, 15 m×0.25 mm ID×0.25 μm film thickness, oven: Tᵢₙᵢ=90° C., ramp to 310° C. at 7° C./min, Tᵢₙⱼ=230° C., T_{det}=250° C., sample preparation: 10 mg/mL in MeCl₂.

### Example 7

### Preparation of (R)-1-(1-methyl-but-2-ynyl)-pyrrolidine

Pyrrolidine (8.00 mL, 96.1 mmol, 2.67 eq) was added to the slurry of the previous step and the mixture was stirred at RT for 18 h. Water (34 g) and aq NaOH (50 wt %, 11.2 g, 141 mmol, 3.92 eq) were added followed by MeCl₂ (10 mL). The resulting phases were separated and the aqueous fraction was washed with MeCl₂ (20 mL). The organic fractions were combined and dried over MgSO₄ and concentrated to an oil. Pentane (23 g) was added and the resulting slurry clarified. The filtrate was concentrated to give (R)-1-(1-methyl-but-2-ynyl)-pyrrolidine as an oil (4.075 g, 82.5 wt %). ¹H NMR (400 MHz, CDCl₃) δ 1.31 (d, J=7 Hz, 3 H), 1.81 (m, 4 H), 2.56 (m, 2 H), 2.64 (m, 2 H), 3.47 (q, J=7 Hz, 1 H); ¹³C NMR (CDCl₃) δ 3.33, 21.40, 23.31, 49.30, 49.74, 78.53, 79.35; GC t_{R}=2.94 min, column: DB-1, 15 m×0.25 mm ID×0.25 μm film thickness, oven: Tᵢₙᵢ=90° C., ramp to 310° C. at 7° C./min, Tᵢₙⱼ=230° C., T_{det}=250° C., sample preparation: 10 mg/mL in MeCl₂; MS (ESI+) for C₉H₁₅N m/z 138 (M+H, 100).

### Example 8

### Preparation of (R)-1-(1-methyl-but-2-ynyl)-pyrrolidine, di-p-toluoyl-L-tartaric acid salt (1:1)

Di-p-toluoyl-L-tartaric acid (3.53 g, 9.13 mmol, 1.00 eq) was added to (R)-1-(1-methyl-but-2-ynyl)-pyrrolidine (1.253 g, 9.13 mmol) in MeCl₂ (20 mL). The resulting solution was concentrated in vacuo to give a slurry (18.8 g). Toluene (20 g) was added followed by ISOPAR C (10 g). The precipitate was collected by vacuum filtration, washed with a mixture of toluene (10 mL) and ISOPAR C (10 mL) and dried in a nitrogen stream to give (R)-1-(1-methyl-but-2-ynyl)-pyrrolidine, di-p-toluoyl-L-tartaric acid salt (1:1, 4.655 g, 97.4%). ¹H NMR (400 MHz, DMSO-d6) δ 1.29 (d, J=7 Hz, 3 H), 1.72 (bs, 4 H), 1.81 (s, 3 H), 2.36 (s, 6 H), 3.03 (bs, 4 H), 4.12 (q, J=7 Hz, 1 H), 5.65 (s, 2 H), 7.34 (d, J=8 Hz, 4 H), 7.84 (d, J=8 Hz, 4 H); ¹³C NMR (DMSO-d6) δ 3.06, 18.84, 21.22, 23.01, 49.64, 50.05, 72.30, 74.1, 83.97, 126.71, 129.33, 129.37, 143.96, 164.91, 168.26; MS (ESI+) for C₉H₁₅N m/z 138 (M+H, 100); [α]²²_{D} (−94.7, C=0.57, MeOH).

### Example 9

### Preparation of (R)-1-(1-methyl-but-2-ynyl)-pyrrolidine (purified)

Aqueous NaOH (50%, 2.07 g, 25.9 mmol, 3.41 eq) was added to a slurry of (R)-1-(1-methyl-but-2-ynyl)-pyrrolidine, di-p-toluoyl-L-tartaric acid salt (1:1, 3.97 g, 7.58 mmol) in water (25 g) and MeCl₂ (42 g). The mixture was warmed to 39° C. and the phases were separated. The organic fraction was washed with water (20 mL) and the aqueous fraction was serial back extracted with MeCl₂ (20 mL). The organic fractions were combined, dried over MgSO₄, and concentrated to give (R)-1-(1-methyl-but-2-ynyl)-pyrrolidine as an oil (0.9085 g, 87.4%). ¹H NMR (400 MHz, CDCl₃) δ 1.23 (d, J=7 Hz, 3 H), 1.69 (m, 4 H), 1.72 (d, J=2 Hz, 3 H), 2.47 (m, 2 H), 2.55 (m, 2 H), 3.38 (m, 1 H); ¹³C NMR (CDCl₃) δ 3.20, 21.30, 23.32, 49.21, 49.63, 78.41, 79.21; GC t_{R}=5.76 min, column: DB-1, 15 m×0.25 mm ID×0.25 μm film thickness, oven: Tᵢₙᵢ=40° C., ramp to 310° C. at 7° C./min, Tᵢₙⱼ=230° C., T_{det}=250° C., sample preparation: 10 mg/mL in MeCl₂; MS (ESI+) for C₉H₁₅N m/z 138 (M+H, 100); [α]²²_{436 nm} (+5.01, C=2.07, EtOAc).

### Example 10

### Preparation of 1-[(1R,2Z)-1-methyl-but-2-en-1-yl]-pyrrolidine

A mixture of (R)-1-(1-methyl-but-2-ynyl)-pyrrolidine (0.150 g, 1.093 mmol), palladium on calcium carbonate (5 wt %, 7.5 mg) and THF (4.5 mL) was hydrogenated at 30° C. and 5 psig for 40 min to afford 1-[(1R,2Z)-1-methyl-but-2-en-1-yl]-pyrrolidine (60 area % by GC, t_{R}=19.57 min) along with starting material, (R)-1-(1-methyl-but-2-ynyl)-pyrrolidine (38 area % by GC, t_{R}=20.68 min). GC conditions: Beta CD 120 column (Supelco), 30 m×0.25 mm ID×0.25 μm film thickness, oven: 70° C. for 15 min, ramp to 220° C. at 20° C./min, hold for 5 min at 220° C., Tᵢₙⱼ=230° C., T_{det}=250° C.

### Example 11

### Preparation of (2E,5S,6E)-5-methyl-3-pyrrolidin-1-yl-octa-2,6-dienoic acid ethyl ester

A mixture of 1-[(1R,2Z)-1-methyl-but-2-en-1-yl]-pyrrolidine (2.254 g, 16.19 mmol), acetonitrile (7.64 g), lithium bromide (1.72 g, 19.78 mmol, 1.22 eq), ethyl 2-butynoate (2.349 g, 20.97 mmol, 1.30 eq) and Et₃N (2.468 g, 24.39 mmol, 1.51 eq) was stirred at 42° C. for 43 h. Toluene (33.47 g) was added and the slurry concentrated (19.90 g). Anhydrous silica gel (2.48 g) was added and the so]ids were removed by vacuum filtration through MgSO₄. The solids were washed with EtOAc in ISOPAR C (15%, 60 mL). The fitrate was concentrated (7 g) and ISOPAR C (30 g) was added. The precipitate was removed by vacuum filtration through MgSO₄ and washed with ISOPAR C and toluene (10 mL). The filtrate was concentrated (4.86 g), ISOPAR C (35 g) was added, and the solution clarified through MgSO₄ with an ISOPAR C rinse. The filtrate was concentrated to give (2E,5S,6E)-5-methyl-3-pyrrolidin-1-yl-octa-2,6-dienoic acid ethyl ester as a yellow oil (3.762 g, 92%). ¹H NMR (400 MHz, CDCl₃) δ 1.06 (d, J=7 Hz, 3 H), 1.25 (t, J=7 Hz, 3 H), 1.62 (d, J=4 Hz, 3 H), 1.99 (bs, 4 H), 2.49 (p, J=6 Hz, 1 H), 2.62 (bs, 1 H), 3.26 (m, 4 H), 4.08 (m, 2 H), 4.46 (s, 1 H), 5.40 (m, 2 H) (strong NOE between signals at 4.46 and 3.26 ppm); ¹³C NMR (CDCl₃) δ 14.73, 17.85, 19.93, 25.19, 36.72, 36.79, 48.13, 58.01, 83.64, 123.10, 136.21, 162.38, 168.49; MS (ESI+) for m/z C₁₅H₂₅NO₂ 252 (M+H, 100); GC t_{R}=16.48 min, column: DB-1, 15 m×0.25 mm ID×0.25 μm film thickness, oven: Tᵢₙᵢ=90° C., ramp to 310° C. at 7° C./min, Tᵢₙⱼ=230° C., T_{det}=250° C., sample preparation: 10 mg/mL in MeOH.

### Example 12

### Preparation of (2Z,5S,6E)-3-amino-5-methyl-octa-2,6-dienoic acid ethyl ester

Anhydrous ammonia in EtOH (2.41 M, 75 mL, 181 mmol, 13.0 eq) was added to (2E,5S,6E)-5-methyl-3-pyrrolidin-1-yl-octa-2,6-dienoic acid ethyl ester (3.50 g, 13.87 mmol). The resulting solution was stirred at 50° C. for 24 h and was subsequently concentrated to give (2Z,5S,6E)-3-amino-5-methyl-octa-2,6-dienoic acid ethyl ester as a yellow oil (2.95 g, 108%). ¹H NMR (400 MHz, CDCl₃) δ 1.10 (d, J=7 Hz, 3 H), 1.26 (t, J=7 Hz, 3 H), 1.65 (d, J=6 Hz, 3 H), 1.99 (dd, J=7, 14 Hz, 1 H), 2.14 (dd, J=7, 14 Hz, 1 H), 2.38 (p, J=7 Hz, 1 H), 4.11 (q, J=7 Hz, 2 H), 4.50 (s, 1 H), 5.35 (dd, J=7, 16 Hz, 1 H), 5.46 (dq, J=6, 15 Hz, 1 H), 7.90 (bs, 2 H); ¹³C NMR (CDCl₃) δ 14.58, 17.93, 20.18, 35.64, 44.01, 58.53, 84.38, 124.29, 135.59, 162.42, 170.39; MS (ESI+) for m/z C₁₁H₁₉NO₂ 198 (M+H, 42), 152 (100), 124 (100); GC t_{R}=9.92 min, column: DB-1, 15 m×0.25 mm ID×0.25 μm film thickness, oven: Tᵢₙᵢ=90° C., ramp to 310° C. at 7° C./min, Tᵢₙⱼ=230° C., T_{det}=250° C., sample preparation: 10 mg/mL in MeOH.

### Example 13

### Preparation of (2Z,5S,6E)-3-acetylamino-5-methyl-octa-2,6-dienoic acid ethyl ester

ISOPAR C (5.63 g), acetic anhydride (1.87 g) and pyridine (2.04 g) were added to (2Z,5S,6E)-3-amino-5-methyl-octa-2,6-dienoic acid ethyl ester (2.00 g, 10.14 mmol). The mixture was sealed in a crimp vial and stirred in a 110° C. bath for 17.5 h. The mixture was cooled to RT and water (2.0 mL) was added. The phases were separated and the organic fraction was washed with water (2.5 mL), sulfuric acid (95 wt %, 0.618 g) in water (2.1 mL), and water (2×2.0 mL). The aqueous layers were serial back extracted with ISOPAR C (2.0 mL). The organic fractions were dried over MgSO₄ and concentrated in vacuo to an oil. Column chromatography, eluting with ethyl acetate (0 to 32%) in hexanes, afforded (2Z,5S,6E)-3-acetylamino-5-methyl-octa-2,6-dienoic acid ethyl ester as a colorless oil (1.82 g, 74.9%) silica gel TLC R_{f}=0.49 (15% EtOAc/ISOPAR C, UV);¹H NMR (400 MHz, CDCl₃) δ 1.00 (d, J=7 Hz, 3 H), 1.29 (t, J=7 Hz, 3 H), 1.63 (d, J=6 Hz, 3 H), 2.14 (s, 3 H), 2.45 (p, J=7 Hz, 1 H), 2.63 (dd, J=7, 13 Hz, 1 H), 2.71 (dd, J=7, 13 Hz, 1 H), 4.16 (q, J=7 Hz, 2 H), 4.87 (s, 1 H), 5.32 (dd, J=7, 16 Hz, 1 H), 5.42 (qd, 1 H, J=6, 15 Hz), 11.06 (s, 1 H); ¹³C NMR (CDCl₃) δ 14.22, 17.86, 20.02, 25.38, 35.13, 41.56, 59.86, 97.43, 123.79, 135.62, 157.09, 168.46, 169.18; MS (ESI−) for m/z C₁₃H₂₁NO₃ 238 (M−H, 79), 192 (32), 113 (100); GC t_{R}=11.73 min, column: DB-1, 15 m×0.25 mm ID×0.25 μm film thickness, oven: Tᵢₙᵢ=90° C., ramp to 310° C. at 7° C./min, Tᵢₙⱼ=230° C., T_{det}=250° C., sample preparation: 10 mg/mL in MeOH.

### Example 14

### Preparation of (3S,5R)-3-acetylamino-5-methyl-octanoic acid ethyl ester

A solution of (2Z,5S,6E)-3-acetylamino-5-methyl-octa-2,6-dienoic acid ethyl ester (1.00 g, 4.179 mmol) and [(R)-BINAPINE-Rh-NBD]⁺BF₄⁻(44 mg, 0.042 mmol, 0.01 eq) in MeOH (15 mL) was hydrogenated at 30 psig hydrogen and 30° C. for 26 h. The resulting solution was concentrated to dryness. MeOH (5 mL) and Pd/C (5%, 50% water wet, 0.5 g) were added and the mixture hydrogenated at 30 psig hydrogen and 30° C. for 18 h. The catalyst was removed by vacuum filtration, washed with MeOH, and the filtrate concentrated to dryness to afford (3S,5R)-3-acetylamino-5-methyl-octanoic acid ethyl ester as a yellow oil (0.576 g, 56.6%) GC t_{R}=12.15 min, column: DB-1, 15 m×0.25 mm ID×0.25 μm film thickness, oven: Tᵢₙᵢ=90° C., ramp to 310° C. at 7° C./min, Tᵢₙⱼ=230° C., T_{det}=250° C., sample preparation: 10 mg/mL in MeOH; ¹H NMR (400 MHz, CDCl₃) δ 0.87 (d, J=7 Hz, 3 H), 0.90 (t, J=6 Hz, 3 H), 1.14 (m, 1 H), 1.27 (t, J=7 Hz, 3 H), 1.98 (s, 3 H), 2.48 (dd, J=2,16 Hz, 1H), 2.55 (dd, J=2,16 Hz, 1H), 4.15 (d, J=5 Hz, 2 H), 4.35 (m, 1 H), 6.09 (m, 1 H); ¹³C NMR (CDCl₃) δ 14.15, 14.27, 19.28, 19.93, 23.41, 29.42, 39.21, 39.49, 41.45, 43.90, 60.51, 169.54, 171.98; MS (ESI+) for C₁₃H₂₅NO₃ m/z 266 (M+Na⁺, 15), 244 (M+H⁺, 36), 198 (M-CH₃CH₂O⁺, 100); [α]²²_{D} (−30.6, C=0.874, ethyl acetate).

### Example 15

### Preparation of (3S,5R)-3-amino-5-methyl-octanoic acid hydrochloride

A mixture of (3S,5R)-3-acetylamino-5-methyl-octanoic acid ethyl ester (0.3791 g, 1.558 mmol), HCl (12 M, 10 mL, 120 mmol, 77 eq) and water (10 mL) was stirred in a scaled vial at 110° C. for 42 h. The resulting solution was concentrated to dryness, dispersed in acetonitrile (10 g), and again concentrated to dryness. Acetonitrile (8.78 g) was added to form a precipitate, which was collected by vacuum filtration, washed with acetonitrile, and dried in a nitrogen stream to give a beige solid (0.2784 g, 92%). Marfey's Assay: 96.3% of (3S,5R)-3-amino-5-methyl-octanoic acid hydrochloride, 3.36% (3S,5S) diastereomer, 0.14% (3R,5R) diastereomer. (Marfey's assay procedure: dissolve 20 mg of title compound in 10 mL of water. Sample 250 μL and add in 250 μL Marfey's reagent (4 mg/mL in acetone) and 50 μL NaHCO₃ (1 M). Heat the mixture to 40° C. for 1 h. Sample 250 μL of the mixture and add 30 μL HCl (1 M). Dilute with mobile phase to 500 μl for injection; mobile phase=620 ml of 50 mM triethylamine in water adjusted to pH 3.0 with phosphoric acid and 380 mL acetonitrile; column 4.6×100 mm BDS Hypersil-keystone C18 at 30° C., detection at 340 nm, flow of 2 mL/min; t_{R} (title compound)=6.64 min, t_{R} ((3S,5S) diastereomer)=5.92 min; t_{R} ((3R,5R) diastereomer)=9.49 min.) ¹H NMR (400 MHz, DMSO-d₆) δ 0.83 (d, J=6 Hz, 3 H), 0.84 (t, J=8 Hz, 3 H), 1.06 (m, 1 H), 1.26 (m, 4 H), 1.60 (m, 2 H), 2.53 (dd, J=7, 17 Hz, 1 H), 2.66 (dd, J=6, 17 Hz, 1 H), 8.10 (s, 3 H); ¹³C NMR (DMSO-d₆) δ 14.18, 19.12, 19.22, 27.69, 37.48, 38.78, 39.78, 45.60, 171.63; MS (ESI+) for C₉H₁₉NO₂ m/z 174 (M+H⁺, 100); [α]²²_{D} (−6.31, C=3.30, DMSO).

### Example 16

### Preparation of (S)-methanesulfonic acid 1-ethyl-but-2-ynyl ester

Methanesulfonyl chloride (1.5 mL, 19.38 mmol, 1.27 eq) was added to a solution of (S)-hex-4-yn-3-ol (1.4933 g, 15.21 mmol, from BASF) in MeCl₂ and Et₃N (3.0 mL, 21.52 mmol, 1.42 eq) at −16° C. During the addition of MsCl the mixture was maintained at a temperature less than 12° C. The resulting slurry was stirred at 0° C. for 1 h and a mixture of HCl (1 M, 5 g) and water (6 g) was added. The resulting phases were separated and the aqueous fraction was washed with MeCl₂ (10 mL). The organic fractions were combined and dried over MgSO₄, clarified, and the solids washed with MeCl₂ (10 mL). The filtrate, which contained (S)-methanesulfonic acid 1-ethyl-but-2-ynyl ester, was used in the next step without purification, but could be concentrated in vacuo to afford a near quantitative yield of the methanesulfonate ester as an oil. ¹H NMR (400 MHz, CDCl₃) δ 1.05 (t, J=7 Hz, 3 H), 1.89 (m, 5 H), 3.11 (s, 3 H), 5.10 (m, 1 H); ¹³C NMR (CDCl₃) δ 3.61, 9.22, 29.42, 39.09, 73.82, 74.91, 85.53.

### Example 17

### Preparation of (R)-1-(1-ethyl-but-2-ynyl)-pyrrolidine

Pyrrolidine (3.80 mL, 45.52 mmol, 2.99 eq) was added to the filtrate of step A and the mixture stirred at RT for 6 days. Water (20 mL) and ISOPAR C (20 mL) were added and the pH of the mixture was adjusted to 7.5 with hydrochloric acid. The phases were separated and the organic fractions were washed with water (15 mL). The aqueous layers were serial back extracted with MTBE (15 mL) and the combined organic fractions were concentrated in vacuo to dryness. Aqueous sodium hydroxide solution (1 M, 10 mL) and MTBE (10 mL) were added and the phases separated. The organic fraction was washed with water (10 mL) and the aqueous fraction serial back extracted with MTBE (10 mL). The combined organic fractions were dried over MgSO₄ and concentrated to dryness to give (R)-1-(1-ethyl-but-2-ynyl)-pyrrolidine. ¹H NMR (400 MHz, CDCl₃) δ 1.01 (t, J=8 Hz, 3 H), 1.64 (m, 2 H), 1.77 (bs, 4 H), 1.84 (d, J=2 Hz, 3 H), 2.57 (m, 2 H), 2.67 (m, 2 H), 3.27 (m, 1 H); ¹³C NMR (CDCl₃) δ 3.33, 11.12, 23.29, 28.26, 49.74, 52.70, 56.45, 73.67, 80.15; GC t_{R}=4.16 min, column: DB-1, 15 m×0.25 mm ID×0.25 μm film thickness, oven: Tᵢₙᵢ=90° C., ramp to 310° C. at 7° C./min, Tᵢₙⱼ=230° C., T_{det}=250° C., sample preparation: 10 mg/mL in MeOH.

### Example 18

### Preparation of 1-[(1R,2Z)-1-ethyl-but-2-en-1-yl]-pyrrolidine

A mixture of (R)-1-(1-ethyl-but-2-ynyl)-pyrrolidine (1.53 g, 10.12 mmol), EtOH (46 mL) and palladium on calcium carbonate (5% Pd, 0.077 g, 0.036 mmol, 0.00358 eq) was hydrogenated at 35 psig for 4 h at 30° C. The catalyst was removed by vacuum filtration with a MeOH wash. The filtrate was concentrated to dryness to afford 1-[(1R,2Z)-1-ethyl-but-2-en-1-yl]-pyrrolidine as an oil.

### Example 19

### Preparation of (3S,5R)-3-amino-5-methyl-nonanoic acid

1-[(1R,2Z)-1-Ethyl-but-2-en-1-yl]-pyrrolidine is converted to (3S,5R)-3-amino-5-methyl-nonanoic acid in a manner similar to the process described above for converting 1-[(1R,2Z)-1-methyl-but-2-en-1-yl]-pyrrolidine to (3S,5R)-3-amino-5-methyl-octanoic acid.

### Example 20

### Preparation of (2E,4R,5R,6E)-4,5-dimethyl-3-pyrrolidin-1-yl-octa-2,6-dienoic acid ethyl ester

A mixture of 1-[(1R,2Z)-1-methyl-but-2-en-1-yl]-pyrrolidine (2.254 g, 16.19 mmol), acetonitrile (7.65 g), lithium bromide (1.71 g, 19.64 mmol, 1.21 eq), ethyl 2-pentynoate (2.688 g, 21.31 mmol, 1.32 eq) and Et₃N (2.448 g, 24.20 mmol, 1.49 eq) was stirred at 65° C. for 20 h and then at 70° C. for 23 h. Toluene (32.5 g) was added and the mixture concentrated in vacuo (22.3 g). Anhydrous silica gel (2.6 g) was added. The resulting mixture was clarified through MgSO₄ and rinsed through with EtOAc in ISOPAR C (15%, 60 mL). The filtrate was concentrated in vacuo (7.0 g) and ISOPAR C (35.1 g) was added. The mixture was clarified through MgSO₄ and rinsed through with ISOPAR C. The filtrate was concentrated in vacuo (5.54 g). ISOPAR C (38 g), MTBE (42 g) and pentane (34.5 g) were added and the mixture concentrated to an oil after each addition to give (2E,4R,5R,6E)-4,5-dimethyl-3-pyrrolidin-1-yl-octa-2,6-dienoic acid ethyl ester as a yellow oil (4.35 g, 101%). ¹H NMR (400 MHz, CDCl₃) δ 0.90 (d, J=7 Hz, 3 H), 1.12 (d, J=7 Hz, 3 H), 1.25 (t, J=7 Hz, 3 H), 1.66 (d, J=6 Hz, 3 H), 1.88 (bs, 4 H), 2.27 (m, 2 H), 2.36 (m, 1 H), 3.32 (m, 2 H), 3.37 (m, 2 H), 4.07 (m, 2 H), 4.45 (s, 1 H), 4.61 (m, 1 H), 5.35 (m, 1 H), 5.42 (m, 1 H); ¹³C NMR (CDCl₃) δ 14.71, 16.47, 17.84, 19.30, 25.14, 36.55, 40.60, 49.29, 58.15, 85.54, 124.50, 136.79, 165.85, 169.02; MS (ESI+) for m/z C₁₆H₂₇NO₂ 266 (M+H, 100); GC t_{R}=17.07 min, column: DB-1, 15 m×0.25 mm ID×0.25 μm film thickness, oven: Tᵢₙᵢ=90° C., ramp to 310° C. at 7° C./min, Tᵢₙⱼ=230° C., T_{det}=250° C., sample preparation: 10 mg/mL in MeOH.

### Example 21

### Preparation of (2Z,4R,5R,6E)-3-amino-4,5-dimethyl-octa-2,6-dienoic acid ethyl ester

Anhydrous NH₃ in MeOH (2.0 M, 120 mL, 240 mmol, 15.9 eq) was added to (2E,4R,5R,6E)-4,5-dimethyl-3-pyrrolidin-1-yl-octa-2,6-dienoic acid ethyl ester (4.00 g, 15.07 mmol). The resulting solution was stirred at 40° C. for 24 h. The solution was concentrated to an oil and ISOPAR C was added. The solution was clarified through MgSO₄ and rinsed through with ISOPAR C. The filtrate was concentrated to give (2Z,4R,5R,6E)-3-amino-4,5-dimethyl-octa-2,6-dienoic acid ethyl ester as a yellow oil (3.27 g, 103%). ¹H NMR (400 MHz, CDCl₃) δ 0.97 (d, J=7 Hz, 3 H), 1.08 (d, J=7 Hz, 1 H), 1.27 (t, J=7 Hz, 3 H), 1.67 (d, J=7 Hz, 3 H), 1.85 (p, J=9 Hz, 1 H), 2.09 (q, J=7 Hz, 1 H), 4.11 (q, J=7 Hz, 2 H), 4.53 (s, 1 H), 5.23 (dd, J=9, 15 Hz, 1 H), 5.45 (dq, J=6, 15 Hz, 1 H); ¹³C NMR (CDCl₃) δ 14.54, 17.57, 17.90, 19.31, 41.71, 46.68, 58.50, 82.77, 125.56, 134.50, 167.66, 170.57; MS (ESI+) for m/z C₁₂H₂₁NO₂ 212 (M+H, 24), 166 (100); GC t_{R}=10.89 min, column: DB-1, 15 m×0.25 mm ID×0.25 μm film thickness, oven: Tᵢₙᵢ=90° C., ramp to 310° C. at 7° C./min, Tᵢₙⱼ=230° C., T_{det}=250° C., sample preparation: 10 mg/mL in MeOH.

### Example 22

### Preparation of (2Z,4R,5R,6E)-3-acetylamino-4,5-dimethyl-octa-2,6-dienoic acid ethyl ester

Acetyl chloride (1.35 mL, 18.99 mmol, 1.34 eq) was added to a solution of (2Z,4R,5R,6E)-3-amino-4,5-dimethyl-octa-2,6-dienoic acid ethyl ester (3.00 g, 14.20 mmol) in MeCl₂ (22 mL) and pyridine (1.60 mL, 19.78 mmol, 1.39 eq) at −60° C. The resulting slurry was stirred at 0° C. for 1.5 h and HCl (1M, 7.0 mL, 7 mmol, 0.49 eq) was added. The phases were separated and the aqueous fraction was washed with MeCl₂ (5 mL). The organic fractions were combined and washed with saturated aq sodium bicarbonate (7 mL) which was back extracted with MeCl₂ (5 mL). The organic fractions were combined and dried over MgSO₄ and concentrated to an oil. Column chromatography, eluting with EtOAc (0 to 64%) in hexanes, afforded (2Z,4R,5R,6E )-3-acetylamino-4,5-dimethyl-octa-2,6-dienoic acid ethyl ester as a colorless oil (2.38 g, 66.2%). Silica gel TLC R_{f}=0.58 (17% EtOAc/ISOPAR C, UV); ¹H NMR (400 MHz, CDCl₃) δ 1.00 (d, J=7 Hz, 3 H), 1.04 (d, J=7 Hz, 3 H), 1.30 (t, J=7 Hz, 3 H), 1.65 (d, J=6 Hz, 3 H), 2.15 (s, 3 H), 2.31 (sextet, J=7 Hz, 1 H), 3.80 (p, J=7 Hz, 1 H), 4.17 (m, 2 H), 4.99 (s, 1 H), 5.25 (dd, J=8, 15 Hz, 1 H), 5.39 (dq, J=6, 15 Hz, 1 H), 11.20 (s, 1 H); ¹³C NMR (CDCl₃) δ 14.21, 15.27, 17.92, 19.05, 25.72, 38.93, 40.89, 59.88, 94.44, 125.37, 132.91, 163.91, 168.71, 169.92; MS (ESI+) for m/z C₁₄H₂₃NO₃ 208 (M-EtO, 86), 166 (100); GC t_{R}=12.87 min, column: DB-1, 15 m×0.25 mm ID×0.25 μm film thickness, oven: Tᵢₙᵢ=90° C, ramp to 310° C. at 7° C./min, Tᵢₙⱼ=230° C., T_{det}=25° C., sample preparation: 10 mg/mL in MeOH; [α]²²_{D} (16.08, C=1.0, EtOAc); Anal. Calc'd for C₁₄H₂₃NO₃: C, 66.37; H, 9.15; N, 5.53. Found: C, 66.39; H, 9.14; N, 5.36.

### Example 23

### Preparation of (3R,4R,5R)-3-acetylamino-4,5-dimethyl-octanoic acid ethyl ester

A mixture of (2Z,4R,5R,6E)-3-acetylamino-4,5-dimethyl-octa-2,6-dienoic acid ethyl ester (1.53 g, 6.04 mmol), MeOH (12 mL) and palladium on strontium carbonate (5% Pd, 0.614 g, 0.288 mmol, 0.048 eq) was hydrogenated at 50 psig for 93 h. The catalyst was removed by vacuum filtration with a MeOH wash. The filtrate was concentrated to dryness to afford (3R,4R,5R)-3-acetylamino-4,5-dimethyl-octanoic acid ethyl ester as an oil (1.431 g, 92%). ¹H NMR (400 MHz, CDCl₃) δ 0.84 (d, J=7 Hz, 3 H), 0.90 (d, J=7 Hz, 3 H), 0.91 (d, J=6 Hz, 3 H), 1.06 (m, 4 H), 1.27 (t, J=7 H, 3 H), 1.53 (m, 2 H), 1.98 (s, 3 H), 2.51 (dd, J=5, 16 Hz, 1 H), 2.57 (dd, J=5, 16 Hz, 1 H), 4.16 (m, 2 H), 4.29 (m, 1 H), 5.95 (d, J=8 Hz, 1 H); ¹³C NMR (CDCl₃) δ 10.97, 14.16, 14.35, 18.11, 20.59, 23.51, 33.07, 33.88, 37.32, 41.26, 48.23, 60.54, 169.31, 172.07; MS (ESI+) for m/z C₁₄H₂₇NO₃ 258 (M+H, 41), 212 (89), 170 (100); GC t_{R}=14.06 min, column: DB-1, 15 m×0.25 mm ID×0.25 μm film thickness, oven: Tᵢₙᵢ=90° C., ramp to 310° C. at 7° C./min, Tᵢₙⱼ=230° C., T_{det}=250_{° C.,} sample preparation: 10 mg/mL in MeOH.

### Example 24

### Preparation of (3R,4R,5R)-3-amino-4,5-dimethyl-octanoic acid hydrochloride salt

Hydrochloric acid (37%, 12 g, 120 mmol, 32 eq) and water (10 mL) were added to (3R,4R,5R)-3-acetylamino-4,5-dimethyl-octanoic acid ethyl ester (0.9808 g, 3.81 mmol). The mixture was stirred at 110° C. for 50 h and the resulting mixture was concentrated in vacuo to a solid. The solids were triturated in acetonitrile (14 mL) and the precipitate collected by vacuum filtration, washed with acetonitrile and dried in a nitrogen stream to give (3R,4R,5R)-3-amino-4,5-dimethyl-octanoic acid hydrochloride salt as a solid (0.697 g, 82%). ¹H NMR (400 MHz, CD₃OD) δ 0.92 (t, J=8 Hz, 3 H), 0.96 (d, J=8 Hz, 3 H), 0.98 (d, J=8 Hz, 3 H), 1.09 (m, 1 H), 1.24 (m, 1 H), 1.33 (m, 1 H), 1.44 (m, 1 H), 1.58 (m, 1 H), 1.64 (septet, J=7 Hz, 1 H), 2.66 (dd, J=8, 20 Hz, 1 H), 2.77 (dd, J=4, 16 Hz, 1 H), 3.59 (dd, J=8, 12 Hz, 1 H); ¹³C NMR (CD₃OD) δ 11.33, 15.05, 18.62, 21.65, 34.93, 35.22, 36.74, 42.17, 52.53; MS (ESI+) for m/z C₁₀H₂₁NO₂ 188 (M+H, 83), 155 (83), 128 (100); [α]²²_{D} (30.73, C=1.0, MeOH); Anal. Calc'd for C₁₀H₂₁NO₂.HCl: C, 53.68; H, 9.91: N, 6.26. Found: C, 53.30; H, 9.69; N, 6.23.

### Example 25

### Preparation of (Z)-1-chloro-but-2-ene

A solution of but-2-yn-1-ol (25.0 g, 356.7 mmol) and ethylene diamine (2.15 g, 35.7 mmol, 0.10 eq) in DMF (63 mL) was hydrogenated at 5 psig H₂ and 30° C. in the presence of Lindlar's catalyst (1.25 g, 5 wt %) for 2 h. Thle catalyst was removed by vacuum filtration and washed with DMF (25 mL). NMR indicated complete conversion to (Z)-but-2-en-1-ol (¹H NMR (400 MHz, CDCl₃) δ 1.41 (d, J=6 Hz, 3 H), 3.94 (d, J=6 Hz, 2 H), 5.33 (m, 2 H); ¹³C NMR (CDCl₃) δ 12.67, 57.39, 125.35, 130.01). Methanesulfonyl chloride (53.1 g, 463 mmol, 1.30 eq) was added over a 6 min period. During the MsCl addition, the temperature of the reaction mixture was allowed to increase to 70° C. where it was maintained. Vacuum was applied to 67 mm Hg and the distillate collected with a dry ice trap (vapor temperature of 28° C. to 62° C., still residue temperature of 45° C. to 90° C.) to afford a yellow oil containing (Z)-1-chloro-but-2-ene (33.33 g, 82%, 84% yield) and DMF (18%); GC t_{R} ((Z)-1-chloro-but-2-ene)=1.37 min, column: DB-1, 15 m×0.25 mm ID×0.25 μm film thickness, oven: Tᵢₙᵢ=40° C., ramp to 310° C. at 7° C./min, Tᵢₙⱼ=230° C., T_{det}=250° C., sample preparation: 10 mg/mL in MeCl₂; ¹H NMR (400 MHz, CDCl₃) δ 1.69 (d, J=6 Hz, 3 H), 4.08 (d, J=7 Hz, 2 H), 5.66 (m, 2 H); ¹³C NMR (CDCl₃) δ 12.56, 39.09, 125.96, 129.59.

### Example 26

### Preparation of (S)-1-[(Z)-but-2-enyl]-2-methyl-pyrrolidine

(Z)-1-Chloro-but-2-ene (77 wt % in DMF, 0.814 g, 6.92 mmol, 1.28 eq) and MeCl₂ (5.9 g) were sequentially added to a mixture of (S)-2-methyl-pyrrolidine (0.4615 g, 5.42 mmol), MeCl₂ (10 mL), water (5 mL) and aq NaOH (50 wt %, 0.878 g, 11.0 mmol, 2.02 eq). The mixture was stirred at 23° C. for 20 h. The phases were separated and the aqueous fraction was washed with MeCl₂ (10 mL). The organic fractions were combined and dried over MgSO₄ and were concentrated to a thin slurry. The supernatant was decanted and the crystals washed with pentane. The supernatant and wash were concentrated to give (S)-1-[(Z)-but-2-enyl]-2-methyl-pyrrolidine as an oil (0.6523 g, 86.4%). GC t_{R}=6.66 min, column: DB-1, 15 m×0.25 mm ID×0.25 μm film thickness, oven: Tᵢₙᵢ=40° C., ramp to 310° C. at 7° C./min, Tᵢₙⱼ=230° C., T_{det}=250° C., sample preparation: 10 mg/mL in MeCl₂; ¹H NMR (400 MHz, CDCl₃) δ 1.12 (d, J=6 Hz, 3 H), 1.48 (m, 1 H), 1.66 (d, J=5 Hz, 3 H), 1.66 (m, 1 H), 1.75 (m, 1 H), 1.9 (m, 1 H), 2.08 (d, J=9 Hz, 1 H), 2.13 (d, J=9 Hz, 1 H), 2.29 (m, 1 H), 2.80 (dd, J=6, 13 Hz, 1 H), 3.12 (td, J=2, 10 Hz, 1 H), 3.41 (dd, J=4, 13 Hz, 1 H), 5.58 (m, 2 H); ¹³C NMR (CDCl₃) δ 13.01, 18.97, 21.43, 32.70, 49.78, 54.02, 59.61, 125.86, 127.78; MS (ESI+) for C₉H₁₇N m/z 140 (M+H)⁺; [α]²²_{D} (+12.5, C=2.82, MeOH).

### Example 27

### Preparation of (2E,4R,5R)-4,5-dimethyl-3-[(2S)-2-methyl-pyrrolidin-1-yl]-hepta-2,6-dienoic acid ethyl ester

A mixture of (S)-1-[(2)-but-2-enyl]-2-methyl-pyrrolidine (0.554 g, 3.975 mmol), acetonitrile (1.94 g), lithium bromide (0.4467 g, 5.14 mmol, 1.29 eq), Et₃N (0.636 g, 6.29 mmol, 1.58 eq) and pent-2-ynoic acid ethyl ester (1.028 g, 8.15 mmol, 2.05 eq) was stirred at 70° C. for 22 h. Toluene (13.6 g) was added and the mixture concentrated (14.5 g). Anhydrous silica gel (0.92 g) was added to the resulting slurry and the mixture was clarified through MgSO₄ (3 g) and rinsed through with ISOPAR C (20 mL) followed by 15% EtOAc in ISOPAR C (15 mL). The mixture was concentrated (2.5 g) and ISOPAR C (25 mL) was added. The slurry was clarified through MgSO₄ (3 g), rinsed through with ISOPAR C (25 mL) and concentrated to an oil (1.462 g). Pentane (41.6 g) was added and the solution concentrated to give (2E,4R,5R)-4,5-dimethyl-3-[(2S)-2-methyl-pyrrolidin-1-yl]-hepta-2,6-dienoic acid ethyl ester as an oil (1.20 g, 114%). GC t_{R}=15.0 min, column: DB-1, 15 m×0.25 min ID×0.25 μm film thickness, oven: Tᵢₙᵢ=90° C., ramp to 310° C. at 7° C./min, Tᵢₙⱼ=230° C., T_{det}=250° C., sample preparation: 10 mg/mL in MeCl₂; MS (ESI+) for C₁₆H₂₇NO₂ m/z 266 (M+H)⁺.

### Example 28

### Preparation of (2Z,4R,5R)-3-amino-4,5-dimethyl-hepta-2,6-dienoic acid ethyl ester

A mixture of (2E,4R,5R)-4,5-dimethyl-3-[(2S)-2-methyl-pyrrolidin-1-yl]-hepta-2,6-dienoic acid ethyl ester (41.15 g, 4.335 mmol) and NH₃ in MeOH (2.0 M, 34 mL, 68 mmol, 15.7 eq) was stirred at 40° C. for 19.5 h and at 45° C. for 22.5 h in a sealed vessel. The mixture was cooled to 23° C. and toluene (25 g) was added. The mixture was concentrated (2 g) and ISOPAR C (50 g) was added. The mixture was again concentrated (1.4 g), ISOPAR C (20 g) was added, the mixture was clarified to remove insolubles, and the solution concentrated (1.5 g). Pentane (20 g) was added and the solution concentrated to give (2Z,4R,5R)-3-amino-4,5-dimethyl-hepta-2,6-dienoic acid ethyl ester as an oil (1.01 g, 118%) %). GC t_{R}=8.52 min, column: DB-1, 15 m×0.25 mm id×0.25 μm film thickness, oven: Tᵢₙᵢ=90° C., ramp to 310° C. at 7° C./min, Tᵢₙⱼ=230° C., T_{det}=250° C., sample preparation: 10 mg/mL in MeCl₂; ¹H NMR (400 MHz, CDCl₃) δ 0.95 (d, J=6 Hz, 3 H), 1.04 (d, J=8 Hz, 3 H), 1.20 (t, J=8 Hz, 3 H), 1.84 (pentet, J=8 Hz, 1 H), 2.11 (pentet, J=8 Hz, 1 H), 4.04 (q, J=8 Hz, 2 H), 4.47 (s, 1 H), 4.95 (d, J=8 Hz, 1 H), 4.98 (d, J=12 Hz, 1 H), 5.57 (ddd, J=4, 8, 12 Hz, 1 H); ¹³C NMR (CDCl₃) δ 14.45, 17.41, 18.70, 42.72, 46.16, 58.36, 82.50, 114.85, 141.76, 167.33, 170.46; MS (ESI+) for C₁₁H₁₉NO₂ m/z 198 (M+H)⁺); [α]₂₂^{D} (−1.5, C=1.55, ethyl acetate).

### Example 29

### Preparation of (2Z,4R,5R)-3-acetylamino-4,5-dimethyl-hepta-2,6-dienoic acid ethyl ester

To a mixture of (2Z,4R,5R)-3-amino-4,5-dimethyl-hepta-2,6-dienoic acid ethyl ester (0.9027 g, 4.576 mmol), MeCl₂ (19 g), and pyridine (0.600 mL, 7.42 mmol, 1.62 eq) was added acetyl chloride (0.45 mL, 6.34 mmol, 1.38 eq) while maintaining the temperature of the mixture at less than −9° C. The mixture was stirred at 0° C. for one hour and HCl (1.0 M, 3 mL, 3 mmol, 0.66 eq) was added. The phases were separated and the organic fraction was washed with saturated aq sodium bicarbonate (10 mL). The aqueous fraction was serial back extracted with MeCl₂ (10 mL) and the combined organic fractions were dried over MgSO₄ and concentrated to an oil. Column chromatography, eluting with EtOAc in hexanes (0 to 64%), gave, after combining and concentrating appropriate fractions, (2Z,4R,5R)-3-acetylamino-4,5-dimethyl-hepta-2,6-dienoic acid ethyl ester as a colorless oil (0.488 g,44.5%, 59.5% from allylic amine). GC t_{R}=10.76 min, column: DB-1, 15 m×0.25 mm ID×0.25 μm film thickness, oven: Tᵢₙᵢ=° C., ramp to 310° C. at 7° C./min, Tᵢₙⱼ=230° C., T_{det}=250° C., sample preparation: 10 mg/mL in MeCl₂; ¹H NMR (400 MHz, CDCl₃) δ 0.96 (d, J=7 Hz, 3 H), 1.04 (d, J=7 Hz, 3 H), 1.25 (t, J=7 Hz, 3 H), 2.11 (s, 3 H), 2.35 (q, J=7 Hz, 1 H), 3.81 (pentet, J=7 Hz, 1 H), 4.12 (m, 2 H), 4.90 (s, 1 H), 4.95 (m, 2 H), 5.60 (ddd, 1 H), 11.2 (s, 1H); ¹³C NMR (CDCl₃) δ 14.13, 14.72, 18.29, 25.61, 38.65, 41.59, 59.85, 94.51, 114.87, 140.05, 163.29, 169.47; MS (ESI+) for C₁₃H₂₁NO₃ m/z 198 ((M+H—CH3CO)⁺, 44), 194 ((M+H-EtOH)⁺, 91), 152 (100); MS (ESI−) for C₁₃H₂₁NO₃ m/z 238 ((M−H)⁻, 100).

### Example 30

### Preparation of (3R,4R,5R)-3-acetylamino-4,5-dimethyl-heptanoic acid ethyl ester

A mixture of (2Z,4R,5R)-3-acetylamino-4,5-dimethyl-hepta-2,6-dienoic acid ethyl ester (0.325 g, 1.357 mmol), Pd on alumina (5 wt % Pd, 0.105 g) and MeOH (7.5 mL) was hydrogenated at 50 psig H₂ and 23° C. for 65 h. The catalyst was removed by pressure filtration, washed with MeOH (2×3 mL) and the filtrate concentrated to dryness to afford (3R,4R,5R)-3-acetylamino-4,5-dimethyl-heptanoic acid ethyl ester as a colorless oil (0.298 g, 90.2%). GC t_{R}=12.14 min; column: DB-1, 15 m×0.25 mm ID×0.25 μm film thickness, oven: Tᵢₙᵢ=90° C., ramp to 310° C. at 7° C./min, Tᵢₙⱼ=230° C., T_{det}=250° C., sample preparation: 10 mg/mL in MeCl₂; ¹H NMR (400 MHz, CDCl₃) δ 0.81 (d, J=9 Hz, 3 H), 0.88 (t, J=7 Hz, 3 H), 0.92 (d, J=7 Hz, 3 H), 1.26 (t, J=7 Hz, 3 H), 1.38 (m, 1 H), 1.56 (m, 1 H), 1.98 (s, 3 H), 2.50 (dd, J=5, 16 Hz, 1 H), 2.55 (dd, J=5, 16 Hz, 1 H), 4.14 (m, 2 H), 5.31 (s, 3 H), 5.91 (d, 1 H); ¹³C NMR (CDCl₃) δ 10.99, 11.87, 14.14, 17.50, 23.47, 23.68, 36.05, 37.56, 41.12, 48.13, 60.53, 169.31, 171.98; MS (ESI+) for C₁₃H₂₅NO₃ m/z 244 ((M+H)⁺, 64), 198 ((M+H-EtOH)⁺, 96); [α]²²_{D} (−6.06, C=0.53, EtOAc).

### Example 31

### Preparation of (3R,4R,5R)-3-amino-4,5-dimethyl-heptanoic acid; hydrochloride

Water (10 mL) and HCl (37 wt %, 10 mL, 121 mmol, 109 eq) were added to (3R,4R,5R)-3-acetylamino-4,5-dimethyl-heptanoic acid ethyl ester (0.2469 g, 1.105 mmol). The mixture was stirred in a sealed vial at 108° C. for 20 h. The resulting solution was concentrated to dryness and comprised (Marfey's assay) 0.41% (3R,4S,5S) diastereomer, t_{R}=5.40 min; <0.1% (3R,4S,5R) diastereomer, t_{R}=5.86 min; 5.26% (3R,4R,5S) diastereomer, t_{R}=6.27 min; 78.47% (3R,4R,5R), t_{R}=7.06 min; 11.68% (3S,4R,5R) diastereomer, t_{R}=9.59 min; 0.78% (3S,4R,5S) diastereomer, t_{R}=10.36 min; 0.31% (3S,4S,5R) diastereomer, t_{R}=10.80 min; 3.09% (3S,4S,5S) diastereomer, t_{R}=11.77 min. Acetonitrile (10 mL) was added and the precipitate was collected by vacuum filtration, washed with acetonitrile and dried in a nitrogen stream to give a solid (115.6 mg, 54%). Marfey's assay showed <0.01% (3R,4S,5S) diastereomer; <0.1% (3R,4S,5R) diastereomer; 3.90% (3R,4R,5S) diastereomer; 76.56% (3R,4R,5R) diastereomer; 13.96% (3S,4R,5R) diastereomer; 0.97% (3S,4R,5S) diastereomer; 0.40% (3S,4S,5R) diastereomer; 4.21% (3S,4S,5S) diastereomer. (Marfey's assay procedure: the derivatization with 1-fluoro-2,4-dinitrophenyl-5-L-alanine amide (Marfey's reagent) was carried out in a 1 dram reaction vial. Solutions of 100 μL Marfey's reagent (10 mg/mL in CH₃CN), 250 μL testing sample (2 mg/mL in 1:1 CH₃CN:H₂O) and 50 μL of 1M sodium bicarbonate were mixed in a 1 dram vial. The mixed solution was incubated at 40^{° C.} for 90 min, and after cooling to RT, 50 μL of 1M HCl was added. A 200 μL aliquot was added into 800 μL 1:1 CH₃CN:H₂) solution for injection (10 μL): aqueous phase (A): pipette 2 mL HClO₄ into 950 mL water and 50 mL CH₃CN; organic phase (13): MeOH; mobile phase: premix 725 mL MeOH and 275 mL aqueous phase: Column YMC Pack Pro C18, 150 mm×4.6 mm, 3 μm; 30° C. column temperature; flow rate 1.0 mL/min; UV detection at 238 nm.) ¹H NMR (400 MHz, CD₃OD) δ 0.77 (t, J=4 Hz, 3 H), 0.83 (d, J=8 Hz, 3 H), 0.85 (d, J=8 Hz, 3 H), 0.98 (m, 1 H), 1.35 (m, 2 H), 1.51 (m, 1 H), 2.53 (dd, J=8, 16 Hz, 1 H), 2.63 (dd, J=8, 20Hz, 1 H), 3.48 (q, J=4 Hz, 1 H); ¹³C NMR (CD₃OD) δ 10.91, 11.69, 17.65, 25.22, 36.41, 41.54, 52.10, 173.67; [α]²²_{D} (14.35, C=0.64, MeOH); MS (ESI+) for C₉H₁₉NO₂ m/z 174 (M+H)⁺.

### Example 32

### Preparation of 1-[(1R,2E)-1-methyl-but-2-en-1-yl]-pyrrolidine, di-p-toluoyl-L-tartaric acid salt

To a mixture of diglyme (11.8 g) and LAH (2.4 M in THF, 9.20 mL, 3.0 eq, 22.1 mmol) was added (R)-1-(1-methyl-but-2-ynyl)-pyrrolidine (1.01 g, 7.36 mmol) followed by diglyme (2.15 mL). The mixture was warmed to 117° C., the resulting distillate discarded, and the mixture stirred at 117° C. for 18 h. The mixture was cooled to RT and ice (15 g) was added while maintaining the mixture at a temperature less than 26° C. THF (20 mL) was added the resulting slurry was vacuum filtered. The filter cake was washed with THF (20 g) and the pH of the filtrate was adjusted from 10.27 to 1.3 with HCl (37%, 1.20 g). Toluene (20 mL) was added to the filtrate, the resulting phases separated, and the aqueous fraction washed with hexanes (10 mL). The organic fraction was serial back extracted with water (7 mL) and the pH of the combined aqueous fractions was adjusted from 1.5 to 10.8 with aq NaOH (50%, 2.2 g). The mixture was extracted with MeCl₂ (2×15 mL) and dried over MgSO₄. Di-p-toluoyl-L-tartaric acid (2.48 g, 6.41 mmol, 0.87 eq) was added and the resulting solution concentrated in vacuo to a thick slurry (11.8 g). Toluene (20 g) was added and the precipitate collected by vacuum filtration, washed with ISOPAR C, and dried in a nitrogen stream to afford 1-[(1R,2E)-1-methyl-but-2-en-1-yl]-pyrrolidine, di-p-toluoyl-L-tartaric acid salt as a white solid (1:1, 2.96 g, 76.5%). ¹H NMR (400 MHz, CDCl₃) δ 1.36 (d, J=7 Hz, 3 H), 1.68 (d, J=6 Hz, 3 H), 1.8237 (m, 2 H), 1.99 (m, 2 H), 2.36 (s, 6 H), 2.73 (m, 2 H), 3.61 (m, 3 H), 5.44 (dd, J=9, 15 Hz, 1 H), 5.76d (dq, J=7, 15 Hz, 1 H), 5.84 (s, 2 H), 7.15 (d, J=8 Hz, 4 H), 7.95 (d, J=8 Hz, 4 H); ¹³C NMR (CDCl₃) δ 17.78, 17.83, 21.62, 23.25, 49.42, 51.24, 62.41, 71.95, 125.40, 126.93, 128.86, 130.02, 134.26, 143.56, 165.60, 170.41; MS (ESI+) for C₉H₁₇N m/z 140 (M+H, 100); [α]²²_{D} (−89.56, C=0.46, MeOH).

### Example 33

### Preparation of 1-[(1R,2E)-1-methyl-but-2-en-1-yl]-pyrrolidine

To 1-[(1R,2E)-1-methyl-but-2-en-1-yl]-pyrrolidine, di-p-toluoyl-L-tartaric acid salt (1:1, 298 mg, 0.567 mmol) was added MeCl₂ (1.64 g) and water (2.16 g) followed by aq NaOH (50%, 0.321 g, 4.01 mmol, 7.07 eq). The mixture was warmed to reflux and the phases separated. The aqueous fraction was washed with MeCl₂ (1.80 g) and the combined organic fractions were dried over MgSO₄ (150 mg). The mixture was clarified with a MeCl₂ rinse and the filtrate concentrated to an oil (72.1 mg, 92.5%) GC t_{R} (1-[(1R,2E)-1-methyl-but-2-en-1-yl]-pyrrolidine)=18.87 min, >98%; t_{R} (opposite enantiomer)=18.96 min, <1%; t_{R} ((S,Z) diastereomer)=19.58 min, 0.41%, column: Beta CD 120 (Supelco), 30 m×0.25 mm ID×0.25 μm film thickness, oven: 70° C. for 15 min, ramp to 220° C. at 20° C./min, hold for 5 min at 220° C., Tᵢₙⱼ=230° C., T_{det}=250° C., sample preparation: 10 mg/mL in MTBE; GC t_{R}=2.09 min, column: DB-1, 15 m×0.25 mm ID×0.25 μm film thickness, oven: Tᵢₙᵢ=90° C., ramp to 310° C. at 7° C./min, Tᵢₙⱼ=230° C., T_{det}=250° C., sample preparation: 10 mg/mL in MeOH; ¹H NMR (400 MHz, CDCl₃) δ 1.19 (d, J=6 Hz, 3 H), 1.68 (d, J=6 Hz, 3 H), 1.78 (m, 4 H), 2.54 (m, 4 H), 2.73 (pentet, J=7 Hz, 1 H), 5.48 (dd, J=8, 21 Hz, 1 H), 5.55 (dq, J=6, 21 Hz, 1 H); ¹³C NMR (CDCl₃) δ 17.63, 20.81, 23.30, 51.91, 62.67, 125.44, 134.71; MS (ESI+) for C₉H₁₇N m/z 140 (M+H, 100).

### Example 34

### Preparation of (2E,5R,6E)-5-methyl-3-pyrrolidin-1-yl-octa-2,6-dienoic acid ethyl ester

A mixture of 1-[(1R,2E)-1-methyl-but-2-en-1-yl]-pyrrolidine (free base, from 2.03 g di-p-toluoyl-L-tartrate salt, 3.86 mmol), lithium bromide (0.428 g, 4.93 mmol, 1.28 eq), acetonitrile (1.84 g), Et₃N (0.633 g, 6.26 mmol, 1.62 eq) and but-2-ynoic acid ethyl ester (0.636 g, 5.68 mmol, 1.47 eq) was stirred at 40° C. for 24 h. Toluene (12 mL) was added and the mixture concentrated (10 g). Dry silica gel (0.53 g) was added and the mixture clarified and rinsed with a mixture of EtOAc (3.75 mL) and hexanes (21.3 mL). The mixture was concentrated (5 mL) and ISOPAR C (25 mL) was added. The mixture was clarified through MgSO₄, rinsed with ISOPAR C, and concentrated to an oil. MTBE (35 g) and pentane (32 g) were added and the solution concentrated to an oil after each addition to afford an oil (0.8597 g, 88.6%). GC t_{R} ((2E,5S,6E)-5-methyl-3-pyrrolidin-1-yl-octa-2,6-dienoic acid ethyl ester)=15.22 min; t_{R} ((2E,5R,6Z) diastereomer)=14.97 min (5.8%): column: DB-1, 15 m×0.25 mm ID×0.25 μm film thickness, oven: Tᵢₙᵢ=90° C., ramp to 310° C. at 7° C./min, Tᵢₙⱼ=230° C., T_{det}=250° C., sample preparation: 10 mg/mL in MeOH; ¹H NMR (400 MHz, CDCl₃) δ 1.04 (d, J=7 Hz, 3 H), 1.23 (t, J=7 Hz, 3 H), 1.60 (d, J=4 Hz, 3 H), 1.97 (bs, 4 H), 2.47 (p, J=6 Hz, 1 H), 2.60 (bs, 1 H), 3.24 (m, 4 H), 4.06 (m, 2 H), 4.44 (s, 1 H), 5.39 (m, 2 H); ¹³C NMR (CDCl₃) δ 14.69, 17.80, 19.89, 25.11, 36.68, 36.76, 48.10, 58.00, 83.64, 123.04, 136.18, 162.31, 168.45; MS (ESI+) for m/z C₁₅H₂₅NO₂ 252 (M+H, 100).

### Example 35

### Preparation of (2Z,5R,6E)-3-amino-5-methyl-octa-2,6-dienoic acid ethyl ester

Anhydrous NH₃ in EtOH (2.41 M, 16 mL, 38 mmol, 16 eq) was added to (2E,5R,6E)-5-methyl-3-pyrrolidin-1-yl-octa-2,6-dienoic acid ethyl ester (0.603 g, 2.40 mmol). The resulting solution was stirred at 55° C. for 19 h. The solution was concentrated to give (2Z,5R,6E)-3-amino-5-methyl-octa-2,6-dienoic acid ethyl ester as a yellow oil (0.531 g, 112%). GC t_{R} ((2Z,5R,6E)-3-amino-5-methyl-octa-2,6-dienoic acid ethyl ester)=8.74 min; t_{R} ((2Z,5S,6Z) diastereomer)=8.46 min (5.67%): column DB-1, 15 m×0.25 mm ID×0.25 μm film thickness, oven: Tᵢₙᵢ=90° C., ramp to 310° C. at 7° C./min, Tᵢₙⱼ=230° C., T_{det}=250° C., sample preparation: 10 mg/mL in MeOH.

### Example 36

### Preparation of (2Z,5R,6E)-3-acetylamino-5-methyl-octa-2,6-dienoic acid ethyl ester

ISOPAR C (2.20 g), acetic anhydride (0.41 g, 4.00 mmol, 1.96 eq) and pyridine (0.429 g, 5.43 mmol, 2.65 eq) were added to (2Z,5R,6E)-3-amino-5-methyl-octa-2,6-dienoic acid ethyl ester (0.403 g, 2.04 mmol). The mixture was sealed in a crimp vial and stirred in a 103° C. bath for 19 h. The mixture was cooled to RT, toluene (20 mL) was added, and the solution concentrated to an oil (0.95 g). Column chromatography, eluting with EtOAc (0 to 16%) in hexanes, afforded (2Z,5R,6E)-3-acetylamino-5-methyl-octa-2,6-dienoic acid ethyl ester as a colorless oil (0.27 g, 55.0%). Silica gel TLC, R_{f}=0.58 (15% EtOAc/ISOPAR C, UV); ¹H NMR (400 MHz, CDCl₃) δ 1.00 (d, J=7 Hz, 3 H), 1.29 (t, J=7 Hz, 3 H), 1.63 (d, J=6 Hz, 3 H), 2.14 (s, 3 H), 2.45 (p, J=7 Hz, 1 H), 2.63 (dd, J=7, 13 Hz, 1 H), 2.71 (dd, J=7, 13 Hz, 1 H), 4.16 (q, J=7 Hz, 2 H), 4.87 (s, 1 H), 5.32 (dd, J=7, 16 Hz, 1 H), 5.42 (qd, 1 H, J=6, 15 Hz ), 11.06 (s, 1 H); ¹³C NMR (CDCl₃) δ 14.22, 17.86, 20.02, 25.38, 35.13, 41.56, 59.86, 97.43, 123.79, 135.62, 157.09, 168.46, 169.18. (Note: NMR was consistent with a 94.2:5.8 mixture of the desired 6E isomer to the undesired 6Z isomer. In particular, small resonances in the carbon spectrum at 20.78, 30.15, 41.42, 123.59 and 135.05 ppm are consistent with low level 6Z diastereomer); GC t_{R} ((2Z,5S,6E)-3-acetylamino-5-methyl-octa-2,6-dienoic acid ethyl ester)=10.28 min, t_{R} ((2Z,5R,6Z) diastereomer)=10.04 min (5.82%): column DB-1, 15 m×0.25 mm ID×0.25 μm film thickness, oven: Tᵢₙᵢ=90° C., ramp to 310° C. at 7° C./min, Tᵢₙⱼ=230° C., T_{det}=250° C., sample preparation: 10 mg/mL in MeOH.

### Example 37

### Preparation of (3R,5S)-3-acetylamino-5-methyl-octanoic acid ethyl ester

A solution of (2Z,5R,6E)-3-acetylamino-5-methyl-octa-2,6-dienoic acid ethyl ester (0.154 g, 0.645 mmol) and [(S)-mTCFP-Rh-(COD)]⁺BF₄⁻ (2 mg, 0.00357 mmol, 0.0055 eq) in MeOH (5 mL) was hydrogenated at 30 psig hydrogen and 30° C. for 120 h. The resulting solution was concentrated to dryness to afford a yellow oil (0.114 g, 73.8%). GC t_{R} ((3R,5S)-3-acetylamino-5-methyl-octanoic acid ethyl ester)=9.48 min, column: DB-1, 15 m×0.25 mm ID×0.25 μm film thickness, oven: Tᵢₙᵢ=90° C., ramp to 310° C. at 7° C./min, Tᵢₙⱼ=230° C., T_{det}=250° C., sample preparation: 10 mg/mL in MeOH; GC t_{R} ((3R,5S)-3-acetylamino-5-methyl-octanoic acid ethyl ester)=32.4 min, GC t_{R} ((3R,5R) and (3S,5S) diastereomers)=32.0 min (total=8.86%), column: Gamma Dex 225, 30 m×0.25 mm ID×0.25 μm film thickness, oven: Tᵢₙᵢ=150° C., hold 25 min, ramp to 210° C. at 5° C./min, Tᵢₙⱼ=230° C., T_{det}=250° C., sample preparation: 10 mg/mL in MeOH; ¹H NMR (400 MHz, CDCl₃) δ 0.87 (d, J=7 Hz, 3 H), 0.90 (t, J=6 Hz, 3 H), 1.14 (m, 1 H), 1.27 (t, J=7 Hz, 3 H), 1.98 (s, 3 H), 2.48 (dd, J=2,16 Hz, 1H), 2.55 (dd, J=2,16 Hz, 1H), 4.15 (d, J=5 Hz, 2 H), 4.35 (m, 1 H), 6.09 (m, 1 H); ¹³C NMR (CDCl₃) δ 14.15, 14.27, 19.28, 19.93, 23.41, 29.42, 39.21, 39.49, 41.45, 43.90, 60.51, 169.54, 171.98; MS (ESI+) for C₁₃H₂₅NO₃ m/z 266 (M+Na⁺, 30), 244 (M+H⁺, 15), 198 (M-CH₃CH₂O³⁰ , 100).

### Example 38

### Preparation of (3R,5S)-3-Amino-5-methyl-octanoic acid hydrochloride

A mixture of (3R,5S)-3-acetylamino-5-methyl-octanoic acid ethyl ester (0.1061 g, 0.436 mmol), HCl (12 M, 6.5 mL, 78 mmol, 179 eq) and water (5.9 mL) was stirred in a sealed vial at 110° C. for 22 h. The resulting solution was concentrated to dryness and acetonitrile (10 g) was added. The slurry was concentrated to dryness and pentane (10 g) was added and the slurry concentrated to dryness to give a beige solid (96.8 mg, 92.8%). Marfey's Assay: 0.60% (3S,5R) enantiomer; 1.77% (3S,5S) diastereomer; 8.39% (3R,5R) diastereomer; and 89.2% (3R,5S)-3-amino-5-methyl-octanoic acid hydrochloride. (Marfey's assay procedure: dissolve 20 mg of (3S,5R)-3-amino-5-methyl-octanoic acid hydrochloride in 10 mL of water. Sample 250 μL and add in 250 μL Marfey's reagent (4 mg/mL in acetone) and 50 μL NaHCO₃ (1 M). Heat the mixture to 40° C. for 1 h. Sample 250 μL of the mixture and add 30 μL HCl (1 M). Dilute with mobile phase to 500 μl for injection; mobile phase=620 mL 50 mM Et₃N in water adjusted to pH 3.0 with phosphoric acid and 380 mL acetonitrile; column 4.6×100 mm BDS Hypersil-keystone C18 at 30° C., detection at 340 nm, flow rate of 2 mL/min; t_{R} ((3S,5R) enantiomer)=6.44 min, t_{R} ((5S,3S) diastereomer)=5.75 min; t_{R} ((5R,3R) diastereomer)=10.9 min; t_{R} ((3R,5S)-3-amino-5-methyl-octanoic)=12.13 min.) ¹H NMR (400 MHz, DMSO-d₆) δ 0.83 (d, J=6 Hz, 3 H), 0.84 (t, J=8 Hz, 3 H), 1.06 (m, 1 H), 1.26 (m, 4 H), 1.60 (m, 2 H), 2.53 (dd, J=7, 17 Hz, 1 H), 2.66 (dd, J=6, 17 Hz, 1 H), 8.10 (s, 3 H); ¹³C NMR (DMSO-d₆) δ 14.18, 19.12, 19.22, 27.69, 37.48, 38.78, 39.78, 45.60, 171.63; MS (ESI+) for C₉H₁₉NO₂ m/z 174 (M+H⁺, 100).

As used in this specification and the appended claims, singular articles such as “a,” “an,” and “the,” may refer to one object or to a plurality of objects unless the context clearly indicates otherwise. Thus, for example, reference to a composition containing “a compound” may include a single compound or two or more compounds. Furthermore, the above description is intended to be illustrative and not restrictive. Many embodiments will be apparent to those of skill in the art upon reading the above description. Therefore, the scope of the invention should be determined with reference to the appended claims. The disclosures of all articles and references, including patent applications, granted patents, and publications, are herein incorporated by reference in their entirety and for all purposes.

1. A method of making a compound of Formula 1, 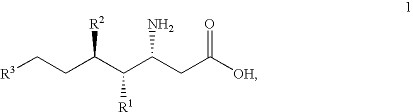
a stereoisomer thereof, or a pharmaceutically acceptable complex, salt, solvate or hydrate of the compound of Formula 1 or the stereoisomer thereof, wherein
R¹, R² and R³ are each independently selected from hydrogen atom, C₁₋₆ alkyl, C₃₋₆ cycloalkyl, C₃₋₆ cycloalkyl-C₁₋₆ alkyl, aryl, aryl-C₁₋₃ alkyl, and arylamino, wherein each alkyl moiety is optionally substituted with from one to five fluorine atoms, and each aryl moiety is optionally substituted with from one to three substituents independently selected from chloro, fluoro, amino, nitro, cyano, C₁₋₃ alkylamino, C₁₋₃ alkyl optionally substituted with from one to three fluorine atoms, and C₁₋₃ alkoxy optionally substituted with from one to three fluorine atoms, provided that R¹ and R² are not both hydrogen atoms;
the method comprising,
reacting a compound of Formula 6, 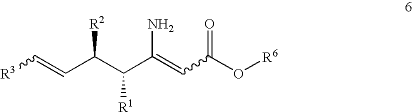
or a compound of Formula 8, 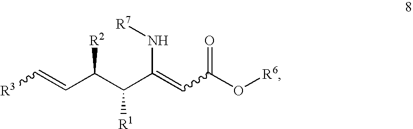
a stereoisomer of the compounds of Formula 6 or Formula 8, or a complex, salt, solvate, or hydrate of the compounds of Formula 6, Formula 8, or the stereoisomer thereof, with H₂ in the presence of a catalyst to give a compound of Formula 9, 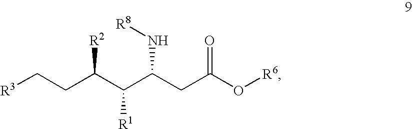
a stereoisomer thereof, or a complex, salt, solvate, or hydrate of the compound of Formula 9 or the stereoisomer thereof, wherein
R¹, R², and R³ in Formula 6, Formula 8, and Formula 9 are as defined for Formula 1;
R⁶ in Formula 6, Formula 8, and Formula 9 is a hydrogen atom, C₁₋₆ alkyl, C₂₋₆ alkenyl, C₂₋₆ alkynyl, C₃₋₇ cycloalkyl, C₃₋₇ cycloalkenyl, halo-C₁₋₇ alkyl, halo-C₂₋₇ alkenyl, halo-C₂₋₇ alkynyl, aryl-C₁₋₆ alkyl, aryl-C₂₋₆ alkenyl, or aryl-C₂₋₆ alkynyl; and
R⁷ in Formula 8 and R⁸ in Formula 9 are each independently selected from hydrogen atom, carboxy, C₁₋₇ alkanoyl, C₂₋₇ alkenoyl, C₂₋₇ alkynoyl, C₃₋₇ cycloalkanoyl, C₃₋₇ cycloalkenoyl, halo-C₁₋₇ alkanoyl, halo-C₂₋₇ alkenoyl, halo-C₂₋₇ alkynoyl, C₁₋₆ alkoxycarbonyl, halo-C₁₋₆ alkoxycarbonyl, C₃₋₇ cycloalkoxycarbonyl, aryl-C₁₋₇ alkanoyl, aryl-C₂₋₇ alkenoyl, aryl-C₂₋₇ alkynoyl, aryloxycarbonyl, and aryl-C₁₋₆ alkoxycarbonyl, provided that R⁷ is not a hydrogen atom;
optionally converting the compound of Formula 9, the stereoisomer thereof, or the complex, salt, solvate or hydrate of the compound of Formula 9 or the stereoisomer thereof, to the compound of Formula 1, the stereoisomer thereof, or the pharmaceutically acceptable complex, salt, solvate or hydrate of the compound of Formula 1 or the stereoisomer thereof,

2. The method of claim 1, wherein the catalyst comprises a chiral phosphine ligand bound to a transition metal through one or more phosphorus atoms.

3. The method of claim 1, further comprising reacting the compound of Formula 6, the stereoisomer thereof, or the complex, salt, solvate, or hydrate of the compound of Formula 6 or the stereoisomer thereof, with a compound of Formula 7,
R⁷—X¹ 7,
to give the compound of Formula 8, the stereoisomer thereof, or the complex, salt, solvate, or hydrate of the compound of Formula 8 or the stereoisomer thereof, wherein R⁷ in Formula 7 is as defined for Formula 8 and X¹ in Formula 7 is a hydroxy or a leaving group.

4. The method of claim 3, wherein X¹ is halogeno, aryloxy, heteroaryloxy, or —OC(O)R⁹, in which R⁹ is C₁₋₆ alkyl, C₂₋₆ alkenyl, C₂₋₆ alkynyl, C₃₋₁₂ cycloalkyl, halo-C₁₋₆ alkyl, halo-C₂₋₆ alkenyl, halo-C₂₋₆ alkynyl, aryl, aryl-C₁₋₆ alkyl, heterocyclyl, heteroaryl, or heteroaryl-C₁₋₆ alkyl.

5. The method of claim 3, further comprising reacting a compound of Formula 5, 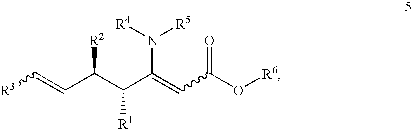
a stereoisomer thereof, or a complex, salt, solvate, or hydrate of the compound of Formula 5 or the stereoisomer thereof, with ammonia to give the compound of Formula 6, the stereoisomer thereof, or the complex, salt, solvate, or hydrate of the compound of Formula 6 or the stereoisomer thereof, wherein R¹, R², and R³ in Formula 5 are as defined for Formula 1, R⁶ is as defined for Formula 6, and R⁴ and R⁵ are each independently selected from C₁₋₆ alkyl, or together with a nitrogen atom to which they are attached, form a 5- or 6-member heterocycle that may be further substituted with none, one, or two substituents selected from C₁₋₆ alkyl.

6. The method of claim 5, further comprising reacting a compound of Formula 2, 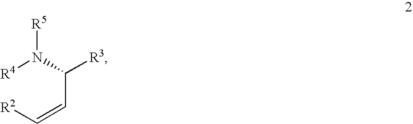
a stereoisomer thereof, or a complex, salt, solvate, or hydrate of the compound of Formula 2 or the stereoisomer thereof, with a compound of Formula 3, 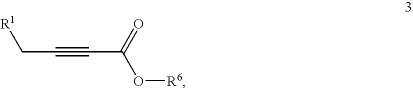
or a complex, salt, solvate, or hydrate thereof, in the presence of a Lewis acid and a base, to give the compound of Formula 5, the stereoisomer thereof, or a complex, salt, solvate, or hydrate thereof, wherein R¹, R², and R³ in Formula 2 and 3 are as defined for Formula 1, R⁴ and R⁵ are as defined for Formula 5, and R⁶ is as defined for Formula 6.

7. A method of making a compound of Formula 5, 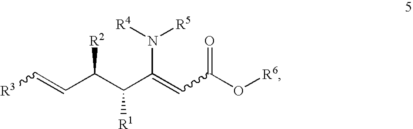
a stereoisomer thereof, or a complex, salt, solvate, or hydrate of the compound of Formula 5 or the stereoisomer thereof, wherein
R¹, R² and R³ are each independently selected from hydrogen atom, C₁₋₆ alkyl, C₃₋₆ cycloalkyl, C₃₋₆ cycloalkyl-C₁₋₆ alkyl, aryl, aryl-C₁₋₃ alkyl, and arylamino, wherein each alkyl moiety is optionally substituted with from one to five fluorine atoms, and each aryl moiety is optionally substituted with from one to three substituents independently selected from chloro, fluoro, amino, nitro, cyano, C₁₋₃ alkylamino, C₁₋₃ alkyl optionally substituted with from one to three fluorine atoms, and C₁₋₃ alkoxy optionally substituted with from one to three fluorine atoms, provided that R¹ and R² are not both hydrogen atoms;
R⁴ and R⁵ are each independently selected from C₁₋₆ alkyl, or together with a nitrogen atom to which R⁴ and R⁵ are attached, form 5- or 6-member heterocycle that may be further substituted with none, one, or two substituents selected from C₁₋₆ alkyl; and
R⁶ is a hydrogen atom, C₁₋₆ alkyl, C₂₋₆ alkenyl, C₂₋₆ alkynyl, C₃₋₇ cycloalkyl, C₃₋₇ cycloalkenyl, halo-C₁₋₇ alkyl, halo-C₂₋₇ alkenyl, halo-C₂₋₇ alkynyl, aryl-C₁₋₆ alkyl, aryl-C₂₋₆ alkenyl, or aryl-C₂₋₆ alkynyl;
the method comprising reacting a compound of Formula 2, 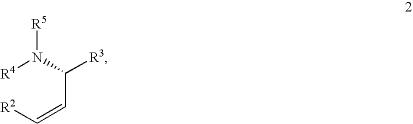
a stereoisomer thereof, or a complex, salt, solvate, or hydrate of the compound of Formula 2 or the stereoisomer thereof, with a compound of Formula 3, 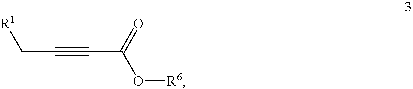
or a complex, salt, solvate, or hydrate thereof, in the presence of a Lewis acid and a base, wherein R¹, R², R³, R⁴, R⁵, and R⁶ in Formula 2 and 3 are as defined for Formula 5.

8. The method of claim 1, wherein R¹ and R² are each independently selected from a hydrogen atom and C₁₋₆ alkyl, and R³ is selected from C₁₋₆ alkyl, C₃₋₆ cycloalkyl, C₃₋₆ cycloalkyl-C₁₋₃ alkyl, phenyl, phenyl-C₁₋₃ alkyl, pyridyl, and pyridyl-C₁₋₃ alkyl, wherein each alkyl is optionally substituted with from one to five fluorine atoms, and each phenyl and pyridyl moiety is optionally substituted with from one to three substituents independently selected from chloro, fluoro, amino, nitro, cyano, C₁₋₃ alkylamino, C₁₋₃ alkyl optionally substituted with from one to three fluorine atoms, and C₁₋₃ alkoxy optionally substituted with from one to three fluorine atoms.

9. The method of claim 1, wherein R¹ is a hydrogen atom or methyl, R² is methyl, and R³ is a hydrogen atom, methyl or ethyl.

10. The method of claim 1, wherein R¹⁰ and R¹¹ are each independently selected from hydrogen atom, C₁₋₆ alkyl, and C₁₋₇ alkanoyl, or together with the nitrogen atom to which they are attached, form pyrrolidine, piperidine, or morpholine rings, which are optionally substituted with none, one, or two substituents selected from C₁₋₆ alkyl.

11. A compound of Formula 10, 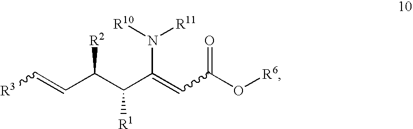
a stereoisomer thereof, or a complex, salt, solvate or hydrate of the compound of Formula 10 or the stereoisomer thereof, wherein
R¹, R² and R³ are each independently selected from hydrogen atom, C₁₋₆ alkyl, C₃₋₆ cycloalkyl, C₃₋₆ cycloalkyl-C₁₋₆ alkyl, aryl, aryl-C₁₋₃ alkyl, and arylamino, wherein each alkyl moiety is optionally substituted with from one to five fluorine atoms, and each aryl moiety is optionally substituted with from one to three substituents independently selected from chloro, fluoro, amino, nitro, cyano, C₁₋₃ alkylamino, C₁₋₃ alkyl optionally substituted with from one to three fluorine atoms, and C₁₋₃ alkoxy optionally substituted with from one to three fluorine atoms, provided that R¹ and R² are not both hydrogen atoms;
R¹⁰ and R¹¹ are each independently selected from hydrogen atom, C₁₋₆ alkyl, carboxy, C₁₋₇ alkanoyl, C₂₋₇ alkenoyl, C₂₋₇ alkynoyl, C₃₋₇ cycloalkanoyl, C₃₋₇ cycloalkenoyl, halo-C₁₋₇ alkanoyl, halo-C₂₋₇ alkenoyl, halo-C₂₋₇ alkynoyl, C₁₋₆ alkoxycarbonyl, halo-C₁₋₆ alkoxycarbonyl, C₃₋₇ cycloalkoxycarbonyl, aryl-C₁₋₇ alkanoyl, aryl-C₂₋₇ alkenoyl, aryl-C₂₋₇ alkynoyl, aryloxycarbonyl, and aryl-C₁₋₆ alkoxycarbonyl, or together with a nitrogen atom to which R¹⁰ and R¹¹ are attached, form a 5- or 6-member heterocycle that may be further substituted with none, one, or two substituents selected from C₁₋₆ alkyl; and
R⁶ is a hydrogen atom, C₁₋₆ alkyl, C₂₋₆ alkenyl, C₂₋₆ alkynyl, C₃₋₇ cycloalkyl, C₃₋₇ cycloalkenyl, halo-C₁₋₇ alkyl, halo-C₂₋₇ alkenyl, halo-C₂₋₇ alkynyl, aryl-C₁₋₆ alkyl, aryl-C₂₋₆ alkenyl, or aryl-C₂₋₆ alkynyl.

12. The compound of claim 11, wherein R¹ and R² are each independently selected from a hydrogen atom and C₁₋₆ alkyl, and R³ is selected from C₁₋₆ alkyl, C₃₋₆ cycloalkyl, C₃₋₆ cycloalkyl-C₁₋₃ alkyl, phenyl, phenyl-C₁₋₃ alkyl, pyridyl, and pyridyl-C₁₋₃ alkyl, wherein each alkyl is optionally substituted with from one to five fluorine atoms, and each phenyl and pyridyl moiety is optionally substituted with from one to three substituents independently selected from chloro, fluoro, amino, nitro, cyano, C₁₋₃ alkylamino, C₁₋₃ alkyl optionally substituted with from one to three fluorine atoms, and C₁₋₃ alkoxy optionally substituted with from one to three fluorine atoms.

13. The compound of claim 11, wherein R¹ is a hydrogen atom or methyl, R² is methyl, and R³ is a hydrogen atom, methyl or ethyl.

14. The compound of claim 11, wherein R¹⁰ and R¹¹ are each independently selected from hydrogen atom, C₁₋₆ alkyl, and C₁₋₇ alkanoyl, or together with the nitrogen atom to which they are attached, form pyrrolidine, piperidine, or morpholine rings, which are optionally substituted with none, one, or two substituents selected from C₁₋₆ alkyl.

15. The compound of claim 11, selected from the following compounds and their complexes, salts, solvates, hydrates, and C₁₋₆ alkyl esters:
(2S,5S)-5-methyl-3-(2-methyl-pyrrolidin-1-yl)-hepta-2,6-dienoic acid,
(S)-5-methyl-3-pyrrolidin-1-yl-octa-2,6-dienoic acid;
(S)-5-methyl-3-pyrrolidin-1-yl-nona-2,6-dienoic acid;
(S)-3-amino-5-methyl-hepta-2,6-dienoic acid;
(S)-3-amino-5-methyl-octa-2,6-dienoic acid;
(S)-3-amino-5-methyl-nona-2,6-dienoic acid;
(S)-3-acetylamino-5-methyl-hepta-2,6-dienoic acid;
(S)-3-acetylamino-5-methyl-octa-2,6-dienoic acid;
(S)-3-acetylamino-5-methyl-nona-2,6-dienoic acid;
(2S,4R,5R)-4,5-dimethyl-3-(2-methyl-pyrrolidin-1-yl)-hepta-2,6-dienoic acid;
(R,R)-4,5-dimethyl-3-pyrrolidin-1-yl-octa-2,6-dienoic acid;
(R,R)-4,5-dimethyl-3-pyrrolidin-1-yl-nona-2,6-dienoic acid;
(R,R)-3-amino-4,5-dimethyl-hepta-2,6-dienoic acid;
(R,R)-3-amino-4,5-dimethyl-octa-2,6-dienoic acid;
(R,R)-3-amino-4,5-dimethyl-nona-2,6-dienoic acid;
(R,R)-3-acetylamino-4,5-dimethyl-hepta-2,6-dienoic acid;
(R,R)-3-acetylamino-4,5-dimethyl-octa-2,6-dienoic acid;
(R,R)-3-acetylamino-4,5-dimethyl-nona-2,6-dienoic acid; and
opposite enantiomers and diastereomers of the aforementioned compounds.
